(19) United States
(12) Patent Application Publication
(10) Pub. No.: US 2008/0075684 A1
(43) Pub. Date: Mar. 27, 2008

(54) Keratin-Binding Polypeptides

(75) Inventors: Heiko Barg, Speyer (DE); Thomas Subkowski, Ladenburg (DE); Hans-Georg Lemaire, Limburgerhof (DE); Claus Bollschweiler, Heidelberg (DE); Arne Ptock, Ludwigshafen (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/597,815

(22) PCT Filed: May 24, 2005
(86) PCT No.: PCT/EP05/05599

§ 371 Date: Nov. 22, 2006

(30) Foreign Application Priority Data
May 24, 2004 (DE) 10 2004 025 805.8
Mar. 14, 2005 (DE) 10 2005 011 988.3

(51) Int. Cl. A61K 8/64; C07K 14/475

(52) U.S. Cl. 424/70.14; 530/350

(57) ABSTRACT

The invention relates to novel keratin-binding protein active substances, and also to the production and use thereof. Particularly suitable keratin-binding polypeptides are sequences which are contained in human desmoplakin or are derived therefrom by altering the human desmoplakin polypeptide sequences such as amino acid insertions, substitutions or deletions. The polypeptide sequences of the human desmoplakin is represented in SEQ ID NO: 1. A suitable keratin-binding domain (domain B) is the polypeptide sequence SEQ ID NO: 1, position 2193 2481, in addition to functional equivalents thereof. An additional keratin-binding domain (domain C) is the polypeptide sequence SEQ ID NO: 1, position 2606 2871 and the functional equivalents thereof.

### PRIOR ART

Vertebrate cells comprise filaments, one group of which is composed of keratins. These keratins also occur in hair, skin and nails, and specific proteins such as, for example, desmoplakin bind thereto by means of a special sequence motif called a keratin-binding domain (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus., Mol Biol Cell. 2003 May; 14(5):1978-92. Epub 2003 Jan. 26; Hopkinson S B, Jones J C., The N terminus of the transmembrane protein BP180 interacts with the N-terminal domain of BP230, thereby mediating keratin cytoskeleton anchorage to the cell surface at the site of the hemidesmosome, Mol Biol Cell. 2000 January; 11(1):277-86).

### OBJECT OF THE INVENTION

It is an object of the present invention to provide novel polypeptides which have a high affinity for keratin or keratin-containing materials such as skin or hair. Such polypeptides are suitable for the cosmetic and pharmaceutical treatment of keratin-containing structures, in particular of hair and skin.

### DESCRIPTION OF THE INVENTION

The invention relates to cosmetic compositions for treating keratin-containing materials, comprising at least one keratin-binding polypeptide sequence (i) in a cosmetically compatible medium.

### Polypeptide Sequences (i)

The polypeptide sequence (i) has a binding affinity for a keratin. The binding of polypeptide sequence (i) to a keratin can be assayed under the conditions described in examples 8, 9 and 10.

Particularly suitable keratin-binding polypeptides are the sequences which are present in human desmoplakin or are derived therefrom by modification of the human desmoplakin polypeptide sequences such as amino acid insertions, substitutions or deletions.

The polypeptide sequence of human desmoplakin is depicted in SEQ ID No: 1. A suitable keratin-binding domain (domain B) is the polypeptide sequence SEQ ID No: 1 position 2193 to 2481, and the functional equivalents thereof. A further keratin-binding domain (domain C) is the polypeptide sequence SEQ ID No: 1 position 2606 to 2871, and the functional equivalents thereof.

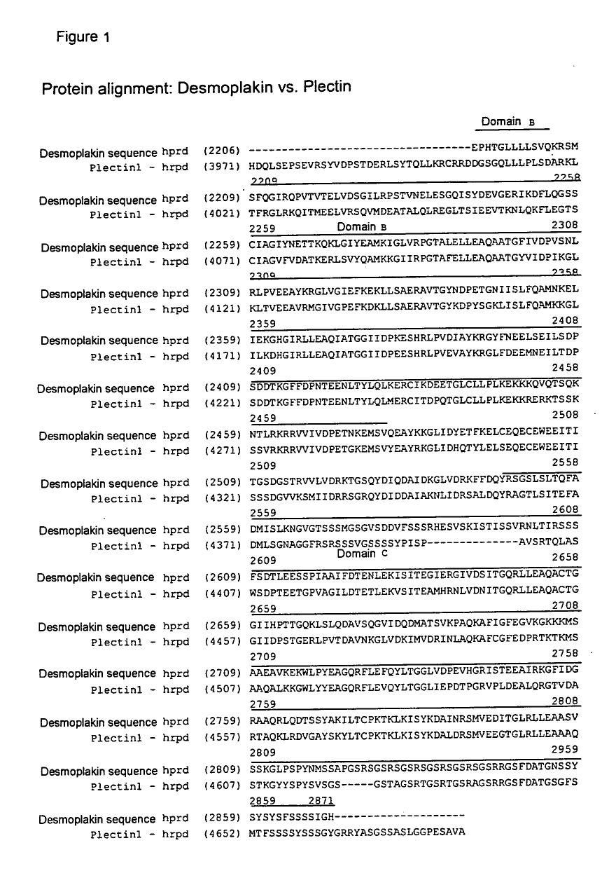

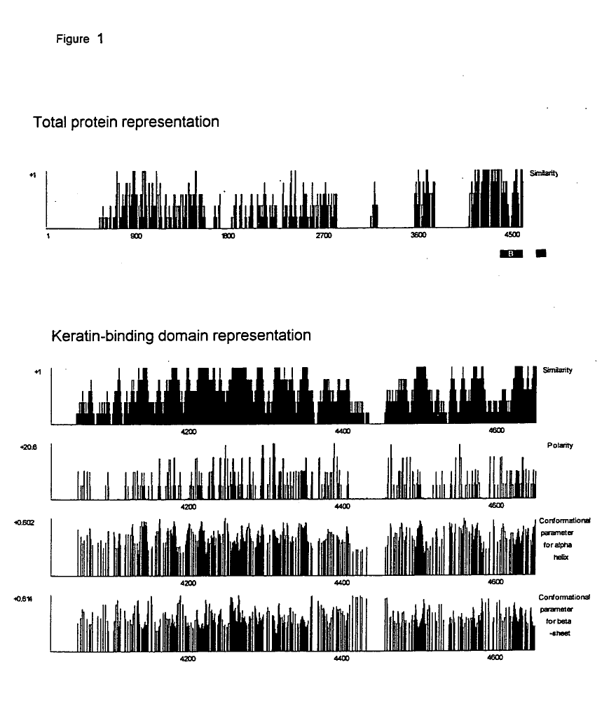

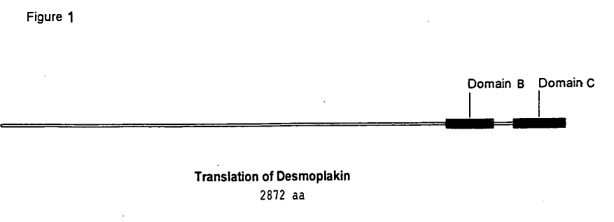

The keratin-binding domains are depicted in FIG. 1.

Preferred polypeptide sequences (i) include an amino acid sequence as shown in SEQ ID No: 1.

Also included according to the invention are likewise “functional equivalents” of the specifically disclosed polypeptide sequences (i) and the use thereof in the methods of the invention.

“Functional equivalents” or analogs of the specifically disclosed polypeptides (i) are for the purposes of the present invention polypeptides which differ therefrom and which additionally have the desired biological activity such as, for example, keratin binding. Thus, for example, “functional equivalents” mean polypeptide sequences which show in one of the binding assays described in example 9 or 10 a binding of at least 10%, preferably at least 50%, particularly preferably 75%, very particularly preferably 90%, of the binding shown by a polypeptide having domain B or domain C of SEQ ID No: 1 in the binding assay described in example 9 or 10.

Examples of suitable amino acid substitutions are to be found in the following table:

| Original residue | Substitution examples |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

It is known that the serine naturally occurring at position 2849 in SEQ ID No: 1 can be replaced for example by glycine in order to avoid phosphorylation at this position (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus., Mol Biol Cell. 2003 May; 14(5):1978-92. Epub 2003 Jan. 26).

“Functional equivalents” mean according to the invention in particular also muteins which have, in at least one sequence position of the abovementioned amino acid sequences, an amino acid other than that specifically mentioned, but nevertheless have one of the abovementioned biological activities. “Functional equivalents” thus include the muteins obtainable by one or more amino acid additions, substitutions, deletions and/or inversions, it being possible for said modifications to occur in any sequence position as long as they lead to a mutein having the property profile according to the invention.

“Functional equivalents” in the above sense are also “precursors” of the described polypeptides, and “functional derivatives” and “salts” of the polypeptides.

“Precursors” are in this connection natural or synthetic precursors of the polypeptides with or without the desired biological activity.

The term “salts” means both salts of carboxyl groups and acid addition salts of amino groups of the protein molecules of the invention. Salts of carboxyl groups can be prepared in a manner known per se and include inorganic salts such as, for example, sodium, calcium, ammonium, iron and zinc salts, and salts with organic bases such as, for example, amines, such as triethanolamine, arginine, lysine, piperidine and the like. The invention likewise relates to acid addition salts such as, for example, salts with mineral acids such as hydrochloric acid or sulfuric acid and salts with organic acids such as acetic acid and oxalic acid.

“Functional derivatives” of polypeptides of the invention can likewise be prepared on functional amino acid side groups or on the N- or C-terminal end thereof by means of known techniques. Such derivatives include for example esters or thioesters of carboxylic acid groups, amides of carboxylic acid groups, obtainable by reaction with ammonia or with a primary or secondary amine; N-acyl derivatives of free amino groups prepared by reaction with acylating agents; N-alkyl derivatives of free amino groups prepared by reaction with alkylating agents; S-acyl derivatives of free mercapto groups prepared by reaction with acylating agents; thioethers by reaction of free mercapto groups with alkylating agents; disulfides by reaction of free mercapto groups, for example with thiols; O-acyl derivatives of free hydroxy groups prepared by reaction with acylating agents; or ethers by reaction of free hydroxyl groups with alkylating agents.

“Functional equivalents” naturally also include polypeptides which are obtainable from other organisms, and naturally occurring variants. It is possible for example to establish ranges of homologous sequence regions by comparison of sequences, and to ascertain equivalent enzymes based on the specific requirements of the invention.

“Functional equivalents” likewise include fragments, preferably single domains or sequence motifs, of the polypeptides of the invention, which have, for example, the desired biological function.

“Functional equivalents” are additionally fusion proteins which comprise one of the abovementioned polypeptide sequences or functional equivalents derived therefrom and at least one further, heterologous sequence which is functionally different therefrom and is in functional N- or C-terminal linkage (i.e. with negligible mutual functional impairment of the parts of the fusion protein). Nonlimiting examples of such heterologous sequences are, for example, signal peptides or enzymes.

“Functional equivalents” also included in the invention are homologs of the specifically disclosed proteins. These have a homology of at least 50%, preferably at least 75%, in particular at least 85%, such as, for example, 90%, 95% or 99%, with one of the specifically disclosed amino acid sequences calculated by the algorithm of Pearson and Lipman, Proc. Natl. Acad, Sci. (USA) 85(8), 1988, 2444-2448. A percentage homology of a homologous polypeptide of the invention means in particular percentage identity of the amino acid residues based on the total length of one of the amino acid sequences specifically described herein.

In the case of possible protein glycosylation, “functional equivalents” of the invention include proteins of the type defined above in deglycosylated or glycosylated form, and modified forms obtainable by altering the glycosylation pattern.

In the case of possible protein phosphorylation, “functional equivalents” of the invention include proteins of the type defined above in dephosphorylated or phosphorylated form, and modified forms obtainable by altering the phosphorylation pattern.

Homologs of the polypeptides (i) of the invention can be generated by mutagenesis, e.g. by point mutation or truncation of the protein.

Homologs of the polypeptides of the invention can be identified by screening combinatorial libraries of mutants, such as, for example, truncation mutants. For example, a library of protein variants can be generated by combinatorial mutagenesis at the nucleic acid level, such as, for example, by enzymatic ligation of a mixture of synthetic oligonucleotides. There is a large number of methods which can be used to prepare libraries of potential homologs from a degenerate oligonucleotide sequence.

Chemical synthesis of a degenerate gene sequence can be carried out in an automatic DNA synthesizer, and the synthetic gene can then be ligated into a suitable expression vector. The use of a degenerate set of genes makes it possible to provide all the sequences which encode the desired set of potential protein sequences in one mixture.

Methods for synthesizing degenerate oligonucleotides are known to the skilled worker (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

Several techniques are known in the art for screening gene products in combinatorial libraries which have been prepared by point mutations or truncation, and for screening cDNA libraries for gene products having a selected property. These techniques can be adapted to the rapid screening of gene libraries which have been generated by combinatorial mutagenesis of homologs of the invention. The most commonly used techniques for screening large gene libraries, which are subject to high-throughput analysis, include the cloning of the gene library into replicable expression vectors, transformation of suitable cells with the resulting vector library and expression of the combinatorial genes under conditions under which detection of the desired activity facilitates isolation of the vector which encodes the gene whose product has been detected. Recursive ensemble mutagenesis (REM), a technique which increases the frequency of functional mutants in the libraries, can be used in combination with the screening tests to identify homologs (Arkin and Yourvan (1992) PNAS 89:7811-7815; Delgrave et al. (1993) Protein Engineering 6(3):327-331).

A particularly advantageous embodiment of the invention are polypeptide sequences (i) which include at least one of the following polypeptide sequences,
a) the polypeptide sequence SEQ ID NO: 1 position 2193 to 248 (domain B)
b) the polypeptide sequence SEQ ID NO: 1 position 2606 to 2871 (domain C)
c) a polypeptide sequence which is modified compared with (a) in up to 60% of the amino acids,
d) a polypeptide sequence which is modified compared with (b) in up to 50% of the amino acids,
   with the proviso that the keratin binding of polypeptide sequence (c) or (d) amounts to at least 10% of the value shown by polypeptide sequence (a) or (b), measured in the assay described in example 9 or 10. Domain B or C means in this connection the keratin-binding domains, described above, of human desmoplakins (SEQ ID No: 1).

Modification of amino acids thereby means amino acid substitutions, insertions and deletions or any combinations of these three possibilities.

Polypeptide sequences (i) preferably used are those having a highly specific affinity for the desired organisms. Accordingly, for applications in skin cosmetics, the polypeptide sequences (i) preferably employed are those having a particularly high affinity for the keratin of human skin. The polypeptide sequences preferred for applications in hair cosmetics are those having a particularly high affinity for the keratin of human hair.

For applications in the pet sector, correspondingly, besides the polypeptide sequences described (SEQ ID NO:1), the preferred polypeptide sequences (i) are those having a particularly high affinity for the corresponding keratin, for example canine keratin or feline keratin.

However, it is also possible to use more than one polypeptide sequence (i) in the effector molecule of the invention, for example a sequence (i) which has a high binding affinity for the keratin of human skin, in conjunction with a sequence (i) which has a high affinity for the keratin of human hair. It is also possible for a plurality of copies of the same polypeptide sequence (i) to be connected consecutively in order, for example, to achieve higher binding.

Suitable keratin-binding polypeptide sequences (i) are known. For example, desmoplakins and plectins comprise keratin-binding domains. (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus., Mol Biol Cell. 2003 May; 14(5):1978-92. Epub 2003 Jan. 26; Hopkinson S B, Jones J C., The N terminus of the transmembrane protein BP180 interacts with the N-terminal domain of BP230, thereby mediating keratin cytoskeleton anchorage to the cell surface at the site of the hemidesmosome, Mol Biol Cell. 2000 January; 11 (1):277-86).

It is possible for such regions to be mapped and identified by alignments of such known protein sequences, for example using a computer program such as Vector NTI 8 (Version of 25 Sep., 2002) supplied by InforMax Inc.

Further suitable polypeptide sequences (i) with good binding to human keratin are sequence regions which show high homology or sequence identity in an alignment and can be regarded as consensus sequences of the keratin-binding domains.

Particular preference is given among these sequence regions to the following:

domain B (KBD-B): polypeptide sequence SEQ ID NO: 1 position 2193 to 2448
domain B (KBD-B): polypeptide sequence SEQ ID NO: 1 position 2209 to 2448
domain C (KBD-C): polypeptide sequence SEQ ID NO: 1 position 2606 to 2871
domain C (KBD-C): polypeptide sequence SEQ ID NO: 1 position 2616 to 2871
domain C (KBD-C): polypeptide sequence SEQ ID NO: 1 position 2616 to 2811
domain C (KBD-C): polypeptide sequence SEQ ID NO: 1 position 2606 to 2871

It is known that the serine naturally occurring at position 2849 in SEQ ID NO: 1 can be replaced for example by glycine in order to avoid phosphorylation at this position and thus to ensure binding of domain C at the corresponding keratin (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus., Mol Biol Cell. 2003 May; 14(5):1978-92. Epub 2003 Jan. 26).

If it is desired that the polypeptide sequences (i) have particularly good binding to a keratin from a non-human organism, the sequence motifs selected as suitable will preferably be those from the keratin-binding protein, e.g. desmoplakin or plectin, of the appropriate organism.

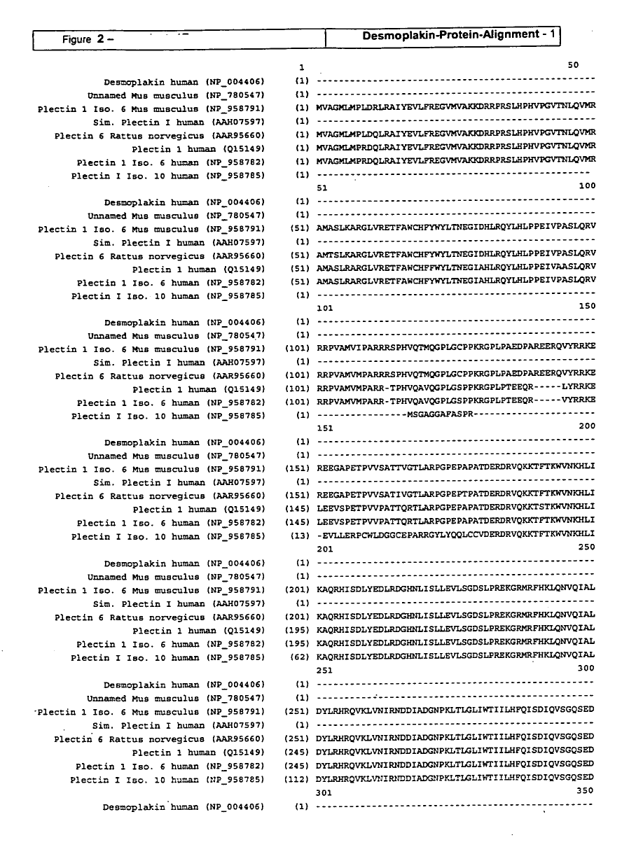

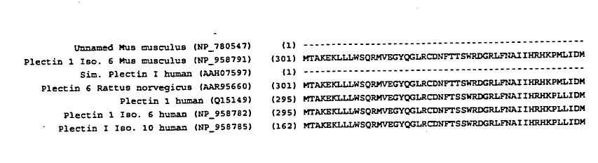

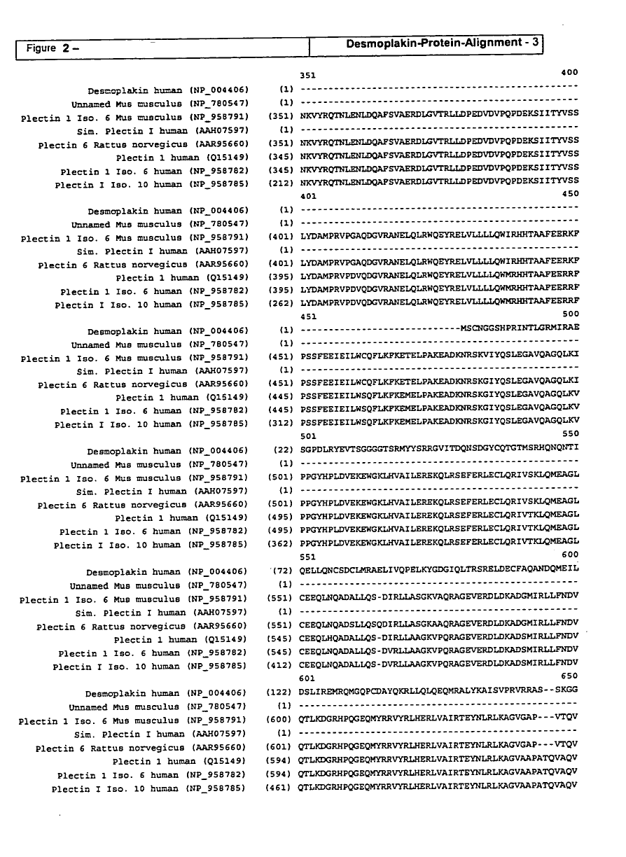

FIG. 2 shows an alignment of keratin-binding molecules.

The keratin-binding polypeptides (i) according to the invention can also, if desired, easily be separated from the keratin again. For this purpose it is possible to employ for example washing with keratin, whereby the keratin-binding polypeptides (i) are displaced from their existing binding to the keratin and are saturated with the keratin from the washing solution. Alternatively, a washing with a high content of detergent (e.g. SDS) is also possible for the washing out.

The keratin-binding polypeptides (i) according to the invention have a wide area of application in human cosmetics, in particular in skin, nail and hair care, animal care, leather care and leather processing.

The keratin-binding polypeptides (i) according to the invention are preferably used for skin cosmetics. They permit a high concentration and long action time of skin care or skin-protecting effectors.

Suitable auxiliaries and additives for producing hair cosmetic, nail cosmetic or skin cosmetic preparations are known to the person skilled in the art and can be found in handbooks of cosmetics, for example Schrader, Grundlagen und Rezepturen der Kosmetika [Fundamentals and formulations of cosmetics], Hüthig Verlag, Heidelberg, 1989, ISBN 3-7785-1491-1.

The cosmetic compositions according to the invention may be skin cosmetic, nail cosmetic, hair cosmetic, dermatological, hygiene or pharmaceutical compositions.

Preferably, the compositions according to the invention are in the form of a gel, foam, spray, ointment, cream, emulsion, suspension, lotion, milk or paste. If desired, liposomes or microspheres can also be used.

The cosmetically or pharmaceutically active compositions according to the invention can additionally comprise cosmetically and/or dermatologically active ingredients and auxiliaries.

Preferably, the cosmetic compositions according to the invention comprise at least one keratin-binding polypeptide sequence (i) as defined above, and at least one constituent different therefrom which is chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tints, tanning agents, dyes, pigments, consistency regulators, moisturizers, re-fatting agents, collagen, protein hydrolysates, lipids, antioxidants, antifoams, antistats, emollients and softeners. The keratin-binding polypeptide active ingredients may also be present in encapsulated form in the cosmetic preparations.

Advantageously, the antioxidants are chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. sodium ascorbate, ascorbyl palmitate, Mg ascorbyl phosphate, ascorbyl acetate), tocopherol and derivatives (e.g. vitamin E acetate, tocotrienol), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

Customary thickeners in such formulations are crosslinked polyacrylic acids and derivatives thereof, polysaccharides and derivatives thereof, such as xanthan gum, agar-agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preference is given to using nonionic thickeners.

Suitable cosmetically and/or dermatologically active ingredients are, for example, coloring active ingredients, skin and hair pigmentation agents, tinting agents, tanning agents, bleaches, keratin-hardening substances, antimicrobial active ingredients, photofilter active ingredients, repellent active ingredients, substances with a hyperemic effect, substances with a keratolytic and keratoplastic effect, antidandruff active ingredient, antiphlogistics, substances with a keratinizing effect, active ingredients with an antioxidative or free-radical-scavenging effect, substances which moisturize the skin or keep the skin moist, re-fatting active ingredients, antierythimatous or antiallergic active ingredients, branched fatty acids such as 18-methyleicosanoic acid, and mixtures thereof.

Active ingredients which tan the skin artificially and which are suitable for tanning the skin without natural or artificial irradiation with UV rays are, for example, dihydroxyacetone, alloxan and walnut shell extract. Suitable keratin-hardening substances are usually active ingredients as are also used in antiperspirants, such as, for example, potassium aluminum sulfate, aluminum hydroxychloride, aluminum lactate, etc.

Antimicrobial active ingredients are used to destroy microorganisms or to inhibit their growth and thus serve both as preservatives and also as deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenic alkylolamides, triethyl citrate, chlorhexidine etc.

Suitable preservatives to be used advantageously according to the invention are listed below with their E number.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | n-Propyl p-hydroxybenzoate |
| E 217 | n-Propyl p-hydroxybenzoate Na salt |
| E 218 | Methyl p-hydroxybenzoate |
| E 219 | Methyl p-hydroxybenzoate Na salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite |
| E 230 | Biphenyl (diphenyl) |
| E 231 | Orthophenylphenol |
| E 232 | Sodium |
| | orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable according to the invention are preservatives or preservative auxiliaries customary in cosmetics dibromodicyanobutane (2-bromo-2-bromomethylglutarodinitrile), 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride and benzyl alcohol. +formaldehyde donors.

Also suitable as preservatives are phenyl hydroxyalkyl ethers, in particular the compound known under the name phenoxyethanol on account of its bactericidal and fungicidal effects on a number of microorganisms.

Other antimicrobial agents are likewise suitable for being incorporated into the preparations according to the invention. Advantageous substances are, for example, 2,4,4′-trichloro-2′-hydroxydiphenyl ether (irgasan), 1,6-di(4-chlorophenylbiguanido)hexane (chlorhexidine), 3,4,4′-trichlorocarbanilide, quaternary ammonium compounds, oil of cloves, mint oil, thyme oil, triethyl citrate, farnesol (3,7,11-trimethyl-2,6,10-dodecatrien-1-ol), and the active ingredients or active ingredient combinations described in the patent laid-open specifications DE-37 40 186, DE-39 38 140, DE-42 04 321, DE42 29 707, DE-43 09 372, DE44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE42 29 737, DE-42 37 081, DE43 24 219, DE44 29 467, DE-44 23 410 and DE-195 16 705. Sodium hydrogencarbonate is also to be used advantageously. Antimicrobial polypeptides can also likewise be used.

Suitable photofilter active ingredients are substances which absorb UV rays in the UV-B- and/or UV-A region. Suitable UV filters are, for example, 2,4,6-triaryl-1,3,5-triazines in which the aryl groups may in each case carry at least one substituent which is preferably chosen from hydroxy, alkoxy, specifically methoxy, alkoxycarbonyl, specifically methoxycarbonyl and ethoxycarbonyl and mixtures thereof. Also suitable are p-aminobenzoic esters, cinnamic esters, benzophenones, camphor derivatives, and pigments which stop UV rays, such as titanium dioxide, talc and zinc oxide.

Suitable UV filter substances are any UV-A and UV-B filter substances. The following examples may be mentioned:

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl) 2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone | 1641-17-4 |
| | (mexenone) | |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3,- | 103597-45-1 |
| | tetramethylbutyl)phenol] | |
| 30 | 2,2′-(1,4-Phenylene)bis-1H-benzimidazole-4,6- | 180898-37-7 |
| | disulfonic acid, Na salt | |
| 31 | 2,4-bis[4-(2-Ethylhexyloxy)-2-hydroxy]phenyl- | 187393-00-6 |
| | 6-(4-methoxyphenyl)-(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone-5,5′- | 3121-60-6 |
| | disodium sulfonate | |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

The cosmetic and dermatological preparations according to the invention may advantageously additionally comprise inorganic pigments which stop UV rays based on metal oxides and/or other metal compounds which are insoluble or slightly soluble in water and chosen from the group of oxides of zinc (ZnO), titanium (TiO₂), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and mixtures of such oxides.

The inorganic pigments can be present here in coated form, i.e. are surface-treated. This surface treatment can consist, for example, in providing the pigments with a thin hydrophobic layer by a method known per se, as described in DE-A-33 14 742.

Suitable repellent active ingredients are compounds which are able to repel or drive away certain animals, in particular insects, from humans. These include, for example, 2-ethyl-1,3-hexanediol, N,N-diethyl-m-toluamide etc. Suitable hyperemic substances, which stimulate the flow of blood through the skin, are e.g. essential oils, such as dwarf pine extract, lavender extract, rosemary extract, juniperberry extract, horse chestnut extract, birch leaf extract, hayflower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiinflammatory agents, which counteract skin irritations, are, for example, allantoin, bisabolol, dragosantol, camomile extract, panthenol, etc.

The cosmetic compositions according to the invention can comprise, as cosmetic and/or pharmaceutical active ingredient (and also if appropriate as auxiliary), at least one cosmetically or pharmaceutically acceptable polymer which differs from the polymers which form the polyelectrolyte complex used according to the invention. These include, quite generally, cationic, amphoteric and neutral polymers.

Suitable polymers are, for example, cationic polymers with the INCI name Poly-quaternium, e.g. copolymers of vinylpyrrolidone/N-vinylimidazolium salts (Luviquat F C, Luviquat H M, Luviquat M S, Luviquat&commat, Care), copolymers of N-vinylpyrrolidone/dimethylaminoethyl methacrylate, quaternized with diethyl sulfate (Luviquat PQ 11), copolymers of N-vinylcaprolactam/N-vinylpyrrolidone/N-vinylimidazolium salts (Luviquat E Hold), cationic cellulose derivatives (Polyquaternium-4 and -10), acrylamido copolymers (Polyquaternium-7) and chitosan.

Suitable cationic (quaternized) polymers are also Merquat (polymer based on dimethyldiallylammonium chloride), Gafquat (quaternary polymers which are produced by the reaction of polyvinylpyrrolidone with quaternary ammonium compounds), Polymer JR (hydroxyethylcellulose with cationic groups) and plant-based cationic polymers, e.g. guar polymers such as the Jaguar grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex 0 Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol 0 Plus (BASF), or polyvinylpyrrolidone and copolymers thereof, in particular with vinyl esters, such as vinyl acetate, e.g. Luviskol 0 VA 37 (BASF), polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/hydroxypropyl methacrylate copolymers obtainable under the names Amphomer (National Starch), and zwitterionic polymers as are disclosed, for example, in the German patent applications DE39 29 973, DE 21 50 557, DE28 17 369 and DE 3708 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are available commercially under the name Amersette (AMERCHOL), and copolymers of hydroxyethyl methacrylate, methyl methacrylate, N,N-dimethylaminoethyl methacrylate and acrylic acid (Jordapon (D)).

Suitable polymers are also nonionic, siloxane-containing, water-soluble or water-dispersible polymers, e.g. polyether siloxanes, such as Tegopren 0 (Goldschmidt) or Besi&commat (Wacker).

The formulation base of pharmaceutical compositions according to the invention preferably comprises pharmaceutically acceptable auxiliaries. Pharmaceutically acceptable auxiliaries are those which are known for use in the field of pharmacy, food technology and related fields, in particular those listed in the relevant pharmacopeia (e.g. DAB Ph. Eur. BP NF) and other auxiliaries whose properties do not preclude a physiological application.

Suitable auxiliaries may be: lubricants, wetting agents, emulsifying and suspending agents, preserving agents, antioxidants, antiirritatives, chelating agents, emulsion stabilizers, film formers, gel formers, odor-masking agents, resins, hydrocolloids, solvents, solubility promoters, neutralizing agents, permeation accelerators, pigments, quaternary ammonium compounds, refatting and superfatting agents, ointment, cream or oil base substances, silicone derivatives, stabilizers, sterilizers, propellants, drying agents, opacifiers, thickeners, waxes, softeners, white oil. Formulation in this regard is based on specialist knowledge, as given, for example, in Fiedler, H. P. Lexikon der Hilfsstoffe für Pharmazie, Kosmetik und angrenzende Gebiete [Lexicon of Auxiliaries for Pharmacy, Cosmetics and related fields], 4th ed., Aulendorf: ECV-Editio-Kantor-Verlag, 1996.

To prepare the dermatological compositions according to the invention, the active ingredients can be mixed or diluted with a suitable auxiliary (excipient). Excipients may be solid, semisolid or liquid materials which can serve as a vehicle, carrier or medium for the active ingredient. Further auxiliaries are added, if desired, in the manner known to the person skilled in the art. In addition, the polymers and dispersions are suitable as auxiliaries in pharmacy, preferably as or in coating(s) or binder(s) for solid drug forms. They can also be used in creams and as tablet coatings and tablet binders.

According to a preferred embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, exfoliating soaps, moisturizing wipes, liquid washing, shower and bath preparations, such as washing lotions, shower baths and gels, foam baths, oil baths and scrub preparations, shaving foams, lotions and creams.

According to a further preferred embodiment, the compositions according to the invention are cosmetic compositions for the care and protection of the skin and hair, nail care compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage makeup, mascara and eye shadows, lipsticks, kohl pencils, eyeliners, blushers, dusting powders and eyebrow pencils.

Furthermore, the polypeptide sequences (i) can be used in nose strips for pore cleansing, in antiacne compositions, repellents, shaving compositions, after-shave and pre-shave care compositions, aftersun care compositions, hair-removal compositions, hair colorants, intimate care compositions, foot care compositions, and in babycare.

The skincare compositions according to the invention are, in particular, W/O or O/w skin creams, day and night creams, eye creams, face creams, antiwrinkle creams, antisun creams, moisturizing creams, bleach creams, self-tanning creams, vitamin creams, skin lotions, care lotions and moisturizing lotions.

Skin cosmetic and dermatological compositions based on the above-described poly-electrolyte complexes exhibit advantageous effects. The polymers can, inter alia, contribute to the moisturization and conditioning of the skin and to an improvement in the feel of the skin. The polymers can also act as thickeners in the formulations. By adding the polymers according to the invention, in certain formulations a considerable improvement in the skin compatibility can be achieved.

Skin cosmetic and dermatological compositions comprise preferably at least one polypeptide sequence (i) in an amount of from about 0.001 to 30% by weight, preferably 0.01 to 20% by weight, very particularly preferably 0.1 to 12% by weight, based on the total weight of the composition.

Particularly photoprotective compositions based on the polypeptide sequences (i) have the property of increasing the residence time of the UV-absorbing ingredients compared to customary auxiliaries such as polyvinylpyrrolidone.

Depending on the field of use, the compositions according to the invention can be applied in a form suitable for skincare, such as, for example, as a cream, foam, gel, stick, mousse, milk, spray (pump spray or propellant-containing spray) or lotion.

Besides the polypeptide sequences (i) and suitable carriers, the skin cosmetic preparations can also comprise further active ingredients and auxiliaries customary in skin cosmetics, as described above. These include preferably emulsifiers, preservatives, perfume oils, cosmetic active ingredients, such as phytantriol, vitamin A, E and C, retinol, bisabolol, panthenol, photoprotective agents, bleaches, colorants, tints, tanning agents, collagen, protein hydrolysates, stabilizers, pH regulators, dyes, salts, thickeners, gel formers, consistency regulators, silicones, moisturizers, re-fatting agents and further customary additives.

Preferred oil and fat components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of the C6-C30-fatty acids, wax esters, such as, for example, jojoba oil, fatty alcohols, vaseline, hydrogenated lanolin and acetylated lanolin, and mixtures thereof.

The polypeptide sequences (i) according to the invention can also be mixed with conventional polymers if specific properties are to be established.

To establish certain properties, such as, for example, improvement in the feel to the touch, the spreading behavior, the water resistance and/or the binding of active ingredients and auxiliaries, such as pigments, the skin cosmetic and dermatological preparations can additionally also comprise conditioning substances based on silicone compounds.

Suitable silicone compounds are, for example, polyalkylsiloxanes, polyarylsiloxanes, polyarylalkylsiloxanes, polyethersiloxanes or silicone resins.

The cosmetic or dermatological preparations are prepared by customary methods known to the person skilled in the art.

Preferably, the cosmetic and dermatological compositions are in the form of emulsions, in particular water-in-oil (W/O) or oil-in-water (O/W) emulsions.

It is, however, also possible to choose other types of formulations, for example gels, oils, oleogels, multiple emulsions, for example in the form of W/O/W or O/W/O emulsions, anhydrous ointments or ointment bases, etc. Emulsifier-free formulations such as hydrodispersions, hydrogels or a Pickering emulsion are also advantageous embodiments.

The emulsions are prepared by known methods. Besides at least one polypeptide sequence (i), the emulsions generally comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The selection of the additives specific to the type of emulsion and the preparation of suitable emulsions is described, for example, in Schrader, Grundlagen und Rezepturen der Kosmetika [Fundamentals and Formulations of Cosmetics], Hüthig Buch Verlag, Heidelberg, 2nd edition, 1989, third part, which is hereby expressly incorporated by reference.

A suitable emulsion as W/O emulsion, e.g. for a skin cream etc., generally comprises an aqueous phase which is emulsified by means of a suitable emulsifier system in an oil or fat phase. To provide the aqueous phase, a polyelectrolyte complex can be used.

Preferred fat components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil, mineral oils whose distillation start point under atmospheric pressure is about 250° C. and whose distillation end point is 410° C., such as, for example, vaseline oil, esters of saturated or unsaturated fatty acids, such as alkyl myristates, e.g. isopropyl, butyl or cetyl myristate, hexadecyl stearate, ethyl or isopropyl palmitate, octanoic or decanoic acid triglycerides and cetyl ricinoleate.

The fatty phase can also comprise silicone oils soluble in other oils, such as dimethylpolysiloxane, methylphenylpolysiloxane and the silicone glycol copolymer, fatty acids and fatty alcohols.

Besides the polypeptide sequences (i) it is also possible to use waxes, such as, for example, carnauba wax, candelilla wax, beeswax, microcrystalline wax, ozokerite wax and Ca, Mg and Al oleates, myristates, linoleates and stearates.

In addition, an emulsion according to the invention can be in the form of an O/W emulsion. Such an emulsion usually comprises an oil phase, emulsifiers which stabilize the oil phase in the water phase, and an aqueous phase, which is usually present in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one polypeptide sequence (i) and customary anionic surfactants as base surfactants and amphoteric and/or nonionic surfactants as cosurfactants. Further suitable active ingredients and/or auxiliaries are generally chosen from lipids, perfume oils, dyes, organic acids, preservatives and antioxidants, and thickeners/gel formers, skin conditioning agents and moisturizers.

These formulations comprise preferably 2 to 50% by weight, preferably 5 to 40% by weight, particularly preferably 8 to 30% by weight, of surfactants, based on the total weight of the formulation.

In the washing, shower and bath preparations it is possible to use all anionic, neutral, amphoteric or cationic surfactants customarily used in body-cleansing compositions.

Suitable anionic surfactants are, for example, alkyl sulfates, alkyl ether sulfates, alkyl-sulfonates, alkylarylsulfonates, alkyl succinates, alkyl sulfosuccinates, N-alkoyl-sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium tauryt sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

Furthermore, the washing, shower and bath preparations can comprise customary cationic surfactants, such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and preservatives, further active ingredients and auxiliaries and water.

According to a further preferred embodiment, the compositions according to the invention are a hair-treatment composition.

Hair-treatment compositions according to the invention preferably comprise at least one polypeptide sequence (i) in an amount in the range from about 0.01 to 30% by weight, preferably 0.5 to 20% by weight, based on the total weight of the composition.

Preferably, the hair-treatment compositions according to the invention are in the form of a setting foam, hair mousse, hair gel, shampoo, hair spray, hair foam, end fluids, neutralizers for permanent waves, hair colorants and bleaches or hot-oil treatments. Depending on the field of use, the hair cosmetic preparations can be applied as (aerosol) spray, (aerosol) foam, gel, gel spray, cream, lotion or wax. Hair sprays here comprise both aerosol sprays and also pump sprays without propellant gas. Hair foams comprise both aerosol foams and also pump foams without propellant gas. Hair sprays and hair foams comprise preferably predominantly or exclusively water-soluble or water-dispersible components. If the compounds used in the hair sprays and hair foams according to the invention are water-dispersible, they can be applied in the form of aqueous microdispersions with particle diameters of from usually 1 to 350 nm, preferably 1 to 250 nm. The solids contents of these preparations here are usually in a range from about 0.5 to 20% by weight. These microdispersions generally require no emulsifiers or surfactants for their stabilization.

The hair cosmetic formulations according to the invention comprise, in a preferred embodiment, a) 0.01 to 30% by weight of at least one polypeptide sequence (i), b) 20 to 99.95% by weight of water and/or alcohol, c) 0 to 50% by weight of at least one propellant gas, d) 0 to 5% by weight of at least one emulsifier, e) 0 to 3% by weight of at least one thickener, and up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, inferface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolysates, alpha- and beta-hydroxycarboxylic acids, stabilizers, pH regulators, dyes, viscosity regulators, gel formers, salts, moisturizers, re-fatting agents, complexing agents and further customary additives.

These also include all styling and conditioner polymers known in cosmetics which can be used in combination with the polypeptide sequences (i) according to the invention if very specific properties are to be established.

Suitable conventional hair cosmetic polymers are, for example, the above-mentioned cationic, anionic, neutral, nonionic and amphoteric polymers, which are hereby incorporated by reference.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and aminofunctional silicone compounds, such as amodimethicones (CTFA).

The polymers according to the invention are particularly suitable as setting agents in hair styling preparations, in particular hair sprays (aerosol sprays and pump sprays without propellant gas) and hair foams (aerosol foams and pump foams without propellant gas).

In a preferred embodiment, spray preparations comprise a) 0.01 to 30% by weight of at least one polypeptide sequence (i), b) 20 to 99.9% by weight of water and/or alcohol, c) 0 to 70% by weight of at least one propellant, d) 0 to 20% by weight of further constituents.

Propellants are the propellants customarily used for hair sprays or aerosol foams. Preference is given to mixtures of propane/butane, pentane, dimethyl ether, 1,1-difluoroethane (HFC-152 a), carbon dioxide, nitrogen or compressed air.

A formulation for aerosol hair foams preferred according to the invention comprises a) 0.01 to 30% by weight of at least one polypeptide sequence (i), b) 55 to 99.8% by weight of water and/or alcohol, c) 5 to 20% by weight of a propellant, d) 0.1 to 5% by weight of an emulsifier, e) 0 to 10% by weight of further constituents.

Emulsifiers which can be used are all of the emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. cetheth-1, polyethylene glycol cetyl ethers, ceteareths, e.g. cetheareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition: a) 0.01 to 30% by weight of at least one polypeptide sequence (i), b) 80 to 99.85% by weight of water and/or alcohol, c) 0 to 3% by weight, preferably 0.05 to 2% by weight, of a gel former, d) 0 to 20% by weight of further constituents.

In general, the polypeptide sequences (i) used according to the invention already have a “self-thickening” effect, meaning that in many cases the use of gel formers can be dispensed with when preparing gels. Their use may, however, be advantageous in order to establish specific rheological or other application properties of the gels. Gel formers which may be used are all gel formers customary in cosmetics. These include slightly crosslinked polyacrylic acid, for example carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) Paraffinum Liquidum (and) PPG-1 trideceth-6, acrylamidopropyltrimonium chloride/acrylamide copolymers, steareth-10 allyl ether, acrylate copolymers, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44.

The polypeptide sequences (i) according to the invention can be used as conditioners in cosmetic preparations.

A preparation comprising the polypeptide sequences (i) according to the invention can preferably be used in shampoo formulations as setting agent and/or conditioner. Preferred shampoo formulations comprise a) 0.01 to 30% by weight of at least one polypeptide sequence (i), b) 25 to 94.95% by weight of water, c) 5 to 50% by weight of surfactants, c) 0 to 5% by weight of a further conditioner, d) 0 to 10% by weight of further cosmetic constituents.

In the shampoo formulations it is possible to use all of the anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Of suitability are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropyl-betaines, alkylsulfobetaines, alkyl glycinates, alkyl carboxyglycinates, alkyl amphoacetates or amphopropionates, alkylamphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, customary conditioners can be used in combination with the polypeptide sequences (i) to achieve certain effects.

These include, for example, the abovementioned cationic polymers with the INCI name Polyquaternium, in particular copolymers of vinylpyrrolidone/N-vinylimidazolium salts (Luviquat FC, Luviquat&commat, HM, Luviquat MS, Luviquat Care), copolymers of N-vinylpyrrolidone/dimethylaminoethyl methacrylate, quaternized with diethyl sulfate (Luviquat D PQ 11), copolymers of N-vinylcaprolactam/N-vinylpyrrolidone/N-vinylimidazolium salts (Luviquat D Hold), cationic cellulose derivatives (Polyquaternium-4 and -10), acrylamide copolymers (Polyquaternium-7). In addition, protein hydrolysates can be used, and conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and aminofunctional silicone compounds such as amodimethicones (CTFA). In addition, cationic guar derivatives, such as guar hydroxypropyltrimonium chloride (INCI) can be used.

The invention further relates to keratin-binding effector molecules consisting of

(i) at least one polypeptide sequence which has a binding affinity for a keratin,
(ii) an effector molecule which is not naturally linked to the polypeptide sequence (i).

Suitable polypeptide sequences (i) are described above.

A particular advantageous embodiment of the invention are polypeptide sequences (i) which include at least one of the following polypeptide sequences,
i. the polypeptide sequence (domain B)
ii. the polypeptide sequence (domain C)
iii. a polypeptide sequence which is modified compared with (a) in up to 70% of the amino acids,
iv. a polypeptide sequence which is modified compared with (b) in up to 70% of the amino acids,
   with the proviso that the keratin binding of polypeptide sequence (c) or (d) amounts to at least 10% of the value shown by polypeptide sequence (a) or (b), measured in the assay described in example 9 or 10. Domain B or C means in this connection the keratin-binding domains, described above and on the following pages, of human desmoplakin (SEQ ID NO: 1). Modification of amino acids thereby means amino acid substitutions, insertions and deletions or any combinations of these three possibilities.

Polypeptide sequences (i) preferably used are those having a highly specific affinity for the desired organism. Accordingly, for applications in skin cosmetics, the polypeptide sequences (i) preferably employed are those having a particularly high affinity for the keratin of human skin. The polypeptide sequences preferred for applications in hair cosmetics are those having a particularly high affinity for the keratin of human hair.

For applications in the pet sector, correspondingly, the preferred polypeptide sequences (i) are those having a particularly high affinity for the corresponding keratin, for example canine keratin or feline keratin.

However, it is also possible to use more than one polypeptide sequence (i) in the effector molecule of the invention, for example a sequence (i) which has a high binding affinity for the keratin of human skin, in conjunction with a sequence (i) which has a high affinity for the keratin of human hair. It is also possible for a plurality of copies of the same polypeptide sequence (i) to be connected consecutively in order, for example, to achieve higher binding.

Suitable keratin-binding polypeptide sequences (i) are known. For example, desmoplakins and plectins comprise keratin-binding domains. (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus., Mol Biol Cell. 2003 May; 14(5):1978-92. Epub 2003 Jan. 26; Hopkinson S B, Jones J C., The N terminus of the transmembrane protein BP180 interacts with the N-terminal domain of BP230, thereby mediating keratin cytoskeleton anchorage to the cell surface at the site of the hemidesmosome, Mol Biol Cell. 2000 January; 11(1):277-86).

It is possible for such regions to be mapped and identified by alignments of such known protein sequences, for example using a computer program such as Vector NTI 8 (Version of Sep. 25, 2002) supplied by InforMax Inc.

Further suitable polypeptide sequences (i) with good binding to human keratin are sequence regions which show high homology or sequence identity in an alignment and can be regarded as consensus sequences of the keratin-binding domains.

Particular preference is given among these sequence regions to the following:

domain B (KBD-B): polypeptide sequence SEQ ID NO: 1 position 2193 to 2448
domain B (KBD-B): polypeptide sequence SEQ ID NO: 1 position 2209 to 2448
domain C (KBD-C): polypeptide sequence SEQ ID NO: 1 position 2606 to 2871
domain C (KBD-C): polypeptide sequence SEQ ID NO: 1 position 2616 to 2871
domain C (KBD-C): polypeptide sequence SEQ ID NO: 1 position 2616 to 2811
domain C (KBD-C): polypeptide sequence SEQ ID NO: 1 position 2606 to 2871

If it is desired that the polypeptide sequences (i) have particularly good binding to a keratin from a non-human organism, the sequence motifs selected as suitable will preferably be those from the keratin-binding protein, e.g. desmoplakin or plectin, of the appropriate organism.

FIG. 2 shows an alignment of keratin-binding molecules.

### Effector Molecules (ii)

Effector molecules (ii) mean hereinafter molecules which have a particular, predictable effect. They may be either proteinaceous molecules such as enzymes or else non-proteinogenic molecules such as dyes, sunscreens, vitamins, provitamins, antioxidants and fatty acids, conditioners, or metal ion-containing compounds.

Among the proteinaceous effector molecules, preference is given to enzymes and antibodies.

Among the enzymes, the following are preferred as effector molecules (ii): oxidases, peroxidases, proteases, glucanases, mutanase, tyrosinases, laccases, metal-binding enzymes, lactoperoxidase, lysozyme, amyloglycosidase, glucose oxidase, superoxide dismutase, photolyase, T4 endonuclease, catalase, thioredoxin, thioredoxin reductase. The proteinaceous effector molecules (ii) without enzymatic activity which are preferred as effector molecules (ii) are the following: antimicrobial peptides, silk proteins, hydrophobins, collaten, carotenoid-binding proteins, heavy metal-binding proteins, odorant-binding proteins.

Also very suitable as proteinaceous effector molecules (ii) are hydrolysates of proteins from vegetable and animal sources, for example hydrolysates of proteins of marine origin or silk hydrolysates.

Among the non-proteinaceous effector molecules (ii), preference is given to dyes, for example food dyes, semipermanent dyes or reactive or oxidation dyes. In the case of oxidation dyes, it is preferred for one component to be coupled as effector molecule (ii) to the keratin-binding polypeptide sequence (i) and then be oxidatively coupled to the second dye component at the site of action, i.e. after binding to the hair. It is further preferred with oxidation dyes to carry out the coupling of the color components before the linkage to the polypeptide sequence (i).

The reactive dyes may further preferably be linked as one component as effector molecule (ii) to the keratin-binding polypeptide sequence (i) and then be bound to the hair. It is further possible for such dyes which are linked as effector molecule (ii) to the keratin-binding polypeptide sequence (i) to be employed in decorative cosmetics through binding to nails or skin.

Suitable dyes for the molecules of the invention are all conventional hair dyes. Suitable dyes are known to the skilled worker from handbooks of cosmetics, for example Schrader, Grundlagen und Rezepturen der Kosmetika, Hüthig Verlag, Heidelberg, 1989, ISBN 3-7785-1491-1.

Particularly advantageous dyes are those specified in the list below. The colour index numbers (CIN) are taken from the Rowe Colour Index, 3^{rd} edition, Society of Dyers and Colourists, Bradford, England, 1971.

| Chemical name or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenylazo)-3-hydroxy-5″- | 12490 | red |
| chloro-2″,4″-dimethoxy-2-naphthanilide | | |
| Disperse Yellow 16 | 12700 | yellow |
| 1-(4-Sulfo-1-phenylazo)-4-aminobenzene-5-sulfonic acid | 13015 | yellow |
| 2,4-Dihydroxyazobenzene-4′-sulfonic acid | 14270 | orange |
| 2-(2,4-Dimethylphenylazo-5-sulfo)-1-hydroxynaphthalene-4-sulfonic | 14700 | red |
| acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfo-4-chloro-5-carboxy-1-phenylazo)-2- | 15525 | red |
| hydroxynaphthalene | | |
| 1-(3-Methylphenylazo-4-sulfo)-2-hydroxynaphthalene | 15580 | red |
| 1-(4′,(8′)-Sulfonaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2- | 15865 | red |
| hydroxynaphthalene-3-carboxylic acid | | |
| 1-(2-Sulfo-1-naphthylazo)-2-hydroxynaphthalene-3-carboxylic | 15880 | red |
| acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5- | 19140 | yellow |
| hydroxypyrazolone-3-carboxylic acid | | |
| Pigment Yellow 16 | 20040 | yellow |
| 2,6-(4′-Sulfo-2″,4″-dimethyl)bisphenylazo)-1,3-dihydroxybenzene | 20170 | orange |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1-hydroxy- | 27755 | black |
| 7-aminonaphthalene-3,6-disulfonic acid | | |
| 4′-[(4″-Sulfo-1″-phenylazo)-7′-sulfo-1′-naphthylazo]-1-hydroxy-8- | 28440 | black |
| acetyl-aminonaphthalene-3,5-disulfonic acid | | |
| Direct Orange 34, 39, 44, 46, 60 | 40215 | orange |
| Food Yellow | 40800 | orange |
| trans-β-Apo-8′-carotenaldehyde (C₃₀) | 40820 | orange |
| trans-Apo-8′-carotenoic acid (C₃₀) ethyl ester | 40820 | orange |
| Canthaxanthine | 40850 | orange |
| Acid Blue 1 | 42045 | blue |
| 2,4-Disulfo-5-hydroxy-4′,4″-bis(diethylamino)triphenylcarbinol | 42051 | blue |
| 4-[(4-N-Ethyl-p-sulfobenzyl(amino)phenyl-(4-hydroxy-2- | 42053 | green |
| sulfophenyl)(methylene)-1-(N-ethyl-N-p-sulfobenzyl)-2,5- | | |
| cyclohexadieneimine] | | |
| Acid Blue 7 | 42080 | blue |
| (N-Ethyl-p-sulfobenzylamino)phenyl-(2-sulfophenyl)methylene- | 42090 | blue |
| (N-ethyl-N-p-sulfobenzyl)-δ^{2,5}-cyclohexadieneimine | | |
| Acid Green 9 | 42100 | green |
| Diethyl disulfobenzyl di-4-amino-2-chlorodi-2- | 42170 | green |
| methylfuchsonimmonium | | |
| Basic Violet 14 | 42510 | violet |
| Basic Violet 2 | 42520 | violet |
| 2′-Methyl-4′-(N-ethyl-N-m-sulfobenzyl)amino-4″-(N- | 42735 | blue |
| diethyl)amino-2-methyl-N-ethyl-N-m- | | |
| sulfobenzylfuchsonimmonium | | |
| 4′-(N-Dimethyl)amino-4″-(N-phenyl)aminonaphtho-N- | 44045 | blue |
| dimethylfuchsonimmonium | | |
| 2-Hydroxy-3,6-disulfo-4,4′- | 44090 | green |
| bisdimethylaminonaphthofuchsonimmonium | | |
| Acid Red 52 | 45100 | red |
| 3-(2′-Methylphenylamino)-6-(2′-methyl-4′-sulfophenylamino)-9- | 45190 | violet |
| (2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinone azine | 69800 | blue |
| Vat Blue 6; Pigment Blue 64 | 69825 | blue |
| Vat Orange 7 | 71105 | orange |
| Indigo | 73000 | blue |
| Indigo disulfonic acid | 73015 | blue |
| 4,4-Dimethyl-6,6′-dichlorothioindigo | 73360 | red |
| 5,5′-Dichloro-7′7-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Natural Yellow 6, 19; Natural Red 1 | 75100 | yellow |
| Bixin, nerbixin | 75120 | orange |
| Lycopene | 75125 | yellow |
| trans-alpha-, beta- or gamma-Carotene | 75130 | orange |
| Keto and/or hydroxyl derivatives of carotene | 75135 | yellow |
| Guanine or pearlescent agent | 75170 | white |
| 1,7-Bis(4-hydroxy-3-methoxyphenyl)-1,6-heptadiene-3,5-dione | 75300 | yellow |
| Complex salt (Na, Al, Ca) of carminic acid | 75470 | red |
| Chlorophyll a and b; copper compounds of chlorophylls and | 75810 | green |
| chlorophyllins | | |
| Aluminum | 77000 | white |
| Hydrated alumina | 77002 | white |
| Hydrous aluminosilicates | 77004 | white |
| Ultramarine Blue | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon Black | 77266 | black |
| Pigment Black 9 | 77267 | black |
| Medicinal charcoal, vegetable carbon | 77268 | black |
| Chromium oxide | 77288 | green |
| Chromium oxide hydrate | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II) and iron(III) hexacyanoferrate | 77510 | blue |
| Pigment white 18 | 77713 | white |
| Manganese ammonium diphosphate | 77742 | violet |
| Manganese phosphate, Mn₃(PO₄)₂•7H₂O | 77745 | red |
| Silver | 77820 | white |
| Titanium dioxide and its mixtures | 77891 | white |
| Zinc oxide | 77947 | white |
| 6,7-Dimethyl-9-(1′-D-ribityl)isoalloxazine, lactoflavin | | yellow |
| Caramel | | brown |
| Capsanthin, capsorubin | | orange |
| Betanin | | red |
| Benzopyrylium salts, anthocyanins | | red |
| Bromocresol Green | | green |
| Aluminum, zinc, magnesium, and calcium stearates | | white |
| Bromothymol Blue | | blue |
| Acid Red 195 | | red |

Also very suitable as hair dyes are food dyes.

The abovementioned dyes can also be used as effector molecules (ii) to a skin- or nail-binding polypeptide sequence (i) for the coloring of skin or nails e.g. in tattoos.

The effector molecule (ii) which are linked to the keratin-binding polypeptide sequence (i) are also, if desired, easily be separated from the keratin in skin, hair or nail again. For this purpose it is possible to employ for example washing with keratin, whereby effector molecule (ii) which are linked to the keratin-binding polypeptide sequence (i) are displaced from their existing binding to the keratin and are saturated with the keratin from the washing solution. Alternatively, a washing with a high content of detergent (e.g. SDS) is also possible for the washing out.

Further preferred effector molecules (ii) are fatty acids, in particular saturated fatty acids carrying an alkyl branch, particularly preferably branched eicosanoic acids, such as 18-methyleicosanoic acid.

Further preferred effector molecules (ii) are carotenoids. According to the invention, carotenoids are understood as meaning the following compounds and esterified or glycosylated derivatives thereof. β-carotene, lycopene, lutein, astaxanthin, zeaxanthin, cryptoxanthin, citranaxanthin, canthaxanthin, bixin, β-apo-4-carotenal, β-apo-8-carotenal, β-apo-8-carotenoic esters, neurosporene, echinenone, adonirubin, violaxanthin, torulene, torularhodin, singly or as mixture. Carotenoids which are preferably used are β-carotene, lycopene, lutein, astaxanthin, zeaxanthin, citranaxanthin and canthaxanthin.

Further preferred effector molecules (ii) are vitamins, especially vitamin A and esters thereof.

Retinoids mean for the purposes of the present invention vitamin A alcohol (retinol) and its derivatives such as vitamin A aldehyde (retinal), vitamin A acid (retinoic acid) and vitamin A esters (e.g. retinyl acetate, retinyl propionate and retinyl palmitate). The term retinoic acid includes in this connection both all-trans-retinoic acid and 13-cis-retinoic acid. The terms retinol and retinal preferably include the all-trans compounds. The retinoid preferably used for the suspensions of the invention is all-trans-retinol, referred to as retinol hereinafter.

Further preferred effector molecules (ii) are vitamins, provitamins and vitamin precursors from the A, C, E and F groups, especially 3,4-didehydroretinol, β-carotene (provitamin of vitamin A), ascorbic acid (vitamin C), and the palmitic esters, glucosides or phosphates of ascorbic acid, tocopherols, especially α-tocopherol and its esters, e.g. the acetate, the nicotinate, the phosphate and the succinate; additionally vitamin F, by which are meant essential fatty acids, especially linoleic acid, linolenic acid and arachidonic acid.

The vitamins, provitamins or vitamin precursors of the vitamin B group or derivatives thereof, and the derivatives of 2-furanone which are preferably to be employed according to the invention include, inter alia:

Vitamin B₁, trivial name thiamine, chemical name 3-[(4′-amino-2′-methyl-5′-pyrimidinyl) methyl]-5-(2-hydroxyethyl)-4-methylthiazolium chloride.

Vitamin B₂, trivial name riboflavin, chemical name 7,8-dimethyl-10-(1-D-ribityl)-benzo[g]pteridine-2,4(3H, 10H)-dione. Riboflavin occurs in free form for example in whey, and other riboflavin derivatives can be isolated from bacteria and yeasts. A riboflavin stereoisomer which is likewise suitable according to the invention is lyxoflavin which can be isolated from fish meal or liver and which has a D-arabityl radical in place of D-ribityl.

Vitamin B₃. The compounds nicotinic acid and nicotinamide (niacinamide) are frequently designated thus. Nicotinamide is preferred according to the invention.

Vitamin B₅ (pantothenic acid and panthenol). Panthenol is preferably employed. Panthenol derivatives which can be employed according to the invention are, in particular, the esters and ethers of panthenol, and cationically derivatized panthenols. In a further preferred embodiment of the invention it is possible to employ in addition to pantothenic acid or panthenol also derivatives of 2-furanone. Particularly preferred derivatives are the substances, which are also commercially available, dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), with all stereoisomers being expressly included.

These compounds advantageously confer moisturizing and skin-soothing properties on the keratin-binding effector molecules of the invention.

Vitamin B₆, by which is meant not a uniform substance but the derivatives of 5-hydroxymethyl-2-methylpyridin-3-ol which are known under the trivial names of pyridoxine, pyridoxamine and pyridoxal.

Vitamin B₇ (biotin), also referred to as vitamin H or “skin vitamin”. Biotin is (3aS, 4S, 6aR)-2-oxohexahydrothienol[3,4-d]imidazole-4-valeric acid.

Panthenol, pantolactone, nicotinamide and biotin are very particularly preferred according to the invention.

It is possible according to the invention to use suitable derivatives (salts, esters, sugars, nucleotides, nucleosides, peptides and lipids). Preferred lipophilic, oil-soluble antioxidants from this group are tocopherol and its derivatives, gallic esters, flavonoids and carotenoids, and butylated hydroxytoluenel/anisole. Preferred water-soluble antioxidants are amino acids, e.g. tyrosine and cysteinee and derivatives thereof, and tannins especially those of vegetable origin.

Triterpenes, especially triterpene acids such as ursolic acid, rosmarinic acid, betulinic acid, boswellic acid and bryonolic acid.

A further preferred effector molecule is lipoic acid and suitable derivatives (salts, esters, sugars, nucleotides, nucleosides, peptides and lipids).

Further preferred effector molecules (ii) are UV light filters. By this are meant organic substances able to absorb ultraviolet rays and emit the absorbed energy again in the form of longer-wavelength radiation, e.g. heat. The organic substances may be oil-soluble or water-soluble.

Examples of oil-soluble UV-B filters which can be used are the following substances:
3-benzylidenecamphor and its derivatives, e.g. 3-(4-methylbenzylidene)camphor;
4-aminobenzoic acid derivatives, preferably 2-ethylhexyl 4-(dimethylamino)benzoate, 2-octyl 4-(dimethylamino)benzoate and amyl 4-(dimethylamino)benzoate; esters of cinnamic acid, preferably 2-ethylhexyl 4-methoxycinnamate, propyl 4-methoxycinnamate, isoamyl 4-methoxycinnamate, isopentyl 4-methoxycinnamate, 2-ethylhexyl 2-cyano-3-phenylcinnamate (octocrylene);
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 2-ethylhexyl 4-methoxybenzalmalonate;
triazine derivatives such as, for example, 2,4,6-trianilino-(p-carbo-2′-ethyl-1′-hexyloxy)-1,3,5-triazine (octyl triazone) and dioctyl butamido triazone (Uvasorb® HEB):
propane-1,3-diones such as, for example, 1-(4-tert-butylphenyl)-3-(4′-methoxyphenyl)propane-1,3-dione.

Suitable water-soluble substances are:
2-phenylbenzimidazole-5-sulfonic acid and the alkali metal, alkaline earth metal, ammonium, alkylammonium, alkanolammonium and glucammonium salts thereof;
sulfonic acid derivatives of benzophenone, preferably 2-hydroxy-4-methoxybenzophenone-5-sulfonic acid and its salts;
sulfonic acid derivatives of 3-benzylidenecamphor such as, for example, 4-(2-oxo-3-bornylidenemethyl)benzenesulfonic acid and 2-methyl-5-(2-oxo-3-bornylidene)sulfonic acid and salts thereof.

It is particularly preferred to use esters of cinnamic acid, preferably 2-ethylhexyl 4-methoxycinnamate, isopentyl 4-methoxycinnamate, 2-ethylhexyl 2-cyano-3-phenylcinnamate (octocrylene).

It is further preferred to use derivatives of benzophenone, in particular 2-hydroxy-4-methoxybenzophenone, 2-hydroxy-4-methoxy-4′-methylbenzophenone, 2,2′-dihydroxy-4-methoxybenzophenone and to use propane-1,3-diones such as, for example 1-(4-tert-butylphenyl)-3-(4′-methoxyphenyl)propane-1,3-dione.

Typical UV-A filters which are suitable are:
derivatives of benzoylmethane such as, for example, 1-(4′-tert-butylphenyl)-3-(4′-methoxyphenyl)propane-1,3-dione, 4-tert-butyl-4′-methoxydibenzoylmethane or 1-phenyl-3-(4′-isopropylphenyl)propane-1,3-dione;
amino-hydroxy-substituted derivatives of benzophenones such as, for example, N,N-diethylaminohydroxybenzoyl-n-hexyl benzoate.

The UV-A and UV-B filters can, of course, also be employed in mixtures.

Further suitable UV filter substances are given in the table below.

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl) 2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone | 1641-17-4 |
| | (mexenone) | |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3,- | 103597-45-1 |
| | tetramethylbutyl)phenol] | |
| 30 | 2,2′-(1,4-Phenylene)bis-1H-benzimidazole-4,6- | 180898-37-7 |
| | disulfonic acid, Na salt | |
| 31 | 2,4-bis[4-(2-Ethylhexyloxy)-2-hydroxy]phenyl- | 187393-00-6 |
| | 6-(4-methoxyphenyl)-(1,3,5)-triazine | |
| 32 | 3-(4-Methylbenzylidene)camphor | 36861-47-9 |
| 33 | Polyethoxyethyl 4-bis(polyethoxy)paraaminobenzoate | 11130-52-9 |
| 34 | 2,4-Dihydroxybenzophenone | 131-56-6 |
| 35 | 2,2′-Dihydroxy-4,4′-dimethoxybenzophenone-5,5′- | 3121-60-6 |
| | disodium sulfonate | |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |

Besides the two aforementioned groups of primary photoprotective substances it is also possible to employ secondary sunscreens of the antioxidant type which break the chain of photochemical reactions which is induced when UV rays penetrate into the skin. Typical examples thereof are superoxide dismutase, catalase, tocopherols (vitamin E) and ascorbic acid (vitamin C).

A further group are anti-irritants which have an anti-inflammatory effect on skin damaged by UV light. Examples of such substances are bisabolol, phytol and phytantriol.

### Linkage of the Effector Molecules (ii) to the Keratin-Binding Polypeptide Sequence (i)

The effector molecules (ii) are connected to a polypeptide sequence (i) which has a binding affinity for a keratin. The connection between (i) and (ii) can be both a covalent bond and based on ionic or van der Waals interactions.

A covalent linkage is preferred. This can take place for example via the side chains of the polypeptide sequence (i), in particular via amino functions or hydroxyl functions or carboxylate functions or thiol functions. Linkage via the amino functions of one or more lysine residues, one or more thiol groups of cysteine residues or via the N-terminal or C-terminal function of the polypeptide (i) is preferred. Apart from the amino acid functions present in the polypeptide sequence (i) it is also possible for amino acids with suitable functions (e.g. cysteines, lysines, aspartates, glutamates) to be attached to the sequence or for amino acids of the polypeptide sequence (i) to be substituted by such amino acid functions.

Linkage of the effector molecules (ii) to the polypeptide sequence (i) can take place either directly, i.e. as covalent linkage of two chemical functions already present in (i) and (ii), for example an amino function of (i) is linked to a carboxylate function of (ii) to give the amide. The linkage can, however, also take place via a so-called linker, i.e. an at least bifunctional molecule, which undergoes bonding with one function to (i) and is linked by one or more other functions to (ii).

If the effector molecule (ii) likewise consists of a polypeptide sequence, the linkage of (i) and (ii) can take place through a so-called fusion protein, i.e. a continuous polypeptide sequence consisting of the two partial sequences (i) and (ii).

It is also possible to incorporate so-called spacer elements between (i) and (ii), for example polypeptide sequences which have a potential cleavage site for a protease, lipase, esterase, phosphatase, hydrolase, or oligo- and polypeptide sequences which allow the fusion protein to be purified easily, for example so-called His tags, i.e. oligohistidine residues.

The spacer elements may further be composed of alkyl chains, ethylene glycol and polyethylene glycols.

Particularly preferred linker and/or spacer elements have a potential cleavage site for a protease, lipase, esterase, phosphatase, hydrolase, i.e. are enzymatically cleavable. Examples of enzymatically cleavable linkers which can be used in the molecules according to the invention are given, for example, in WO 98/01406, to the entire contents of which reference is hereby expressly made.

Particularly preferred linkers and spacers are thermally cleavable, photocleavable. Corresponding chemical structures are known to the person skilled in the art and are integrated between the molecular moieties (i) and (ii).

Linkage in the case of a non-proteinaceous effector molecule to the polypeptide sequence (i) preferably takes place with functionalizable residues (side groups, C or N terminus) on the polypeptide (i) which undergo covalent connection to the chemical function of the effector molecule.

The linkage in this case is preferably via an amino, thiol or hydroxyl function of the polypeptide (i), which are able to undergo a corresponding amide, thioester or ester bonding for example with a carboxyl function of the effector molecule (ii), where appropriate after activation.

A further preferred linkage of the polypeptide sequence (i) to an effector molecule (ii) is the use of a tailored linker. Such a linker has two or more so-called anchor groups with which it can link the polypeptide sequence (i) and one or more effector molecules (ii). For example, an anchor group for (i) may be a thiol function by means of which the linker can undergo disulfide bonding to a cysteine residue of the polypeptide (i). An anchor group for (ii) may be for example a carboxyl function by means of which the linker can undergo ester bonding to a hydroxyl function of the effector molecule (ii).

The use of such tailored linkers allows the linkage to be adapted accurately to the desired effector molecule. It is additionally possible thereby to link a plurality of effector molecules to a polypeptide sequence (i) in a defined manner.

The linker which is used depends on the functionality to be coupled. Suitable examples are molecules which couple to polypeptides (i) by means of sulfhydryl-reactive groups, e.g. maleimides, pyridyl disulfides, α-haloacetyls, vinyl sulfones, sulfatoalkyl sulfones (preferably sulfatoethyl sulfones) and to effector molecules (ii) by means of
sulfhydryl-reactive groups (e.g. maleimides, pyridyl disulfides, α-haloacetyls, vinyl sulfones, sulfatoalkyl sulfones (preferably sulfatoethyl sulfones)
amine-reactive groups (e.g. succinimidyl esters, carbodiimides, hydroxymethylphosphine, imidoesters, PFP esters etc.)
sugars or oxidized sugar-reactive groups (e.g. hydrazides etc.)
carboxy-reactive groups (e.g. carbodiimides etc.)
hydroxyl-reactive groups (e.g. isocyanates etc.)
thymine-reactive groups (e.g. psoralen etc.)
nonselective groups (e.g. aryl azides etc.)
photoactivatable groups (e.g. perfluorophenyl azide etc.)
metal-complexing groups (e.g. EDTA, hexahis, ferritin)
antibodies and fragments thereof (e.g single-chain antibodies, F(ab) fragments of antibodies, catalytic antibodies).

An alternative possibility is direct coupling between active substance/effector and the keratin-binding domain, e.g. by means of carbodiimides, glutaraldehyde, the abovementioned crosslinkers or other crosslinkers known to the skilled worker.

The keratin-binding effector molecules of the invention can also, if desired, easily be separated from the keratin again. It is possible to employ for this purpose for example washing with keratin, whereby the keratin-binding effector molecules are displaced from their existing binding to the keratin and are saturated with the keratin from the washing solution. Reversible adhesion of a plurality of effector molecules to keratin is thus possible. Alternatively, a washing with a high content of detergent (e.g. SDS) is possible for the washing out.

The keratin-binding effector molecules of the invention have a wide area of application in human cosmetics, especially in skin and hair care, animal care, leather care and leather processing.

The keratin-binding effector molecules of the invention are preferably used for skin, nail and hair cosmetics. They permit a high concentration and long duration of action of skin-, nail- and hair-care or skin-, nail- and hair-protecting effectors.

Suitable auxiliaries and additives for producing hair cosmetic or skin cosmetic preparations are familiar to the skilled worker and can be found in handbooks of cosmetics, for example Schrader, Grundlagen und Rezepturen der Kosmetika, Hüthig Verlag, Heidelberg, 1989, ISBN 3-7785-1491-1.

In a further embodiment, this hair cosmetic or skin cosmetic preparation serves to care for or protect the skin or hair and is the form of an emulsion, a dispersion, a suspension, an aqueous surfactant preparation, a milk, a lotion, a cream, a balsam, an ointment, a gel, a granulation, a dusting powder, a stick product such as, for example, a lipstick, a foam an aerosol or a spray. Such formulations are very suitable for topical preparations. Suitable emulsions are oil-in-water emulsions and water-in-oil emulsions or microemulsions.

The hair cosmetic or skin cosmetic preparation is ordinarily used for application to the skin (topically) or hair. Topical preparations mean in this connection preparations which are suitable for applying the active substances to the skin in fine distribution and preferably in a form which can be absorbed through the skin. Examples suitable for this purpose are aqueous and hydroalcoholic solutions, sprays, foams, foam aerosols, ointments, aqueous gels, emulsions of the O/W or W/O type, microemulsions or cosmetic stick products.

In a preferred embodiment of the cosmetic composition of the invention, the composition comprises a carrier. A preferred carrier is water, a gas, a water-based liquid, an oil, a gel, an emulsion or microemulsion, a dispersion or a mixture thereof. Said carriers are well tolerated by skin. Particularly advantageous for topical preparations are aqueous gels, emulsions or microemulsions.

Emulsifiers which can be used are nonionic surfactants, zwitterionic surfactants, ampholytic surfactants or anionic emulsifiers. The emulsifiers may be present in the composition of the invention in amounts of from 0.1 to 10, preferably 1 to 5, % by weight based on the composition.

It is possible to use as nonionic surfactant for example a surfactant from at least one of the following groups:

adducts of 2 to 30 mol of ethylene oxide and/or 0 to 5 mol of propylene oxide with linear fatty alcohols having 8 to 22 C atoms, with fatty acids having 12 to 22 C atoms and with alkylphenols having 8 to 15 C atoms in the alkyl group;

### C_{12/18} fatty acid monoesters and diesters of adducts of 1 to 30 mol of ethylene oxide with glycerol;

glycerol monoesters and diesters and sorbitan monoesters and diesters of saturated and unsaturated fatty acids having 6 to 22 carbon atoms and their ethylene oxide adducts;
alkyl mono- and oligoglycosides having 8 to 22 carbon atoms in the alkyl radical and their ethoxylated analogs;
adducts of 15 to 60 mol of ethylene oxide with castor oil and/or hardened castor oil;
polyol esters and especially polyglycerol esters such as, for example, polyglycerol polyricinoleate, polyglycerol poly-12-hydroxystearate or polyglycerol dimerate. Likewise suitable are mixtures of compounds from a plurality of these substance classes;
adducts of 2 to 15 mol of ethylene oxide with castor oil and/or hardened castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose);
mono-, di- and trialkyl phosphates and mono-, di- and/or tri-PEG-alkyl phosphates and the salts thereof;
wool wax alcohols;
polysiloxane-polyalkyl polyether copolymers and corresponding derivatives;
mixed esters of pentaerythritol, fatty acids, citric acid and fatty alcohol as disclosed in DE 1165574 and/or mixed esters of fatty acids having 6 to 22 carbon atoms, methyl glucose and polyols, preferably glycerol or polyglycerol, and
polyalkylene glycols;
betaines.

Zwitterionic surfactants can also be used as emulsifiers. The surface-active compounds referred to as zwitterionic surfactants are those having at least one quaternary ammonium group and at least one carboxylate or one sulfonate group in the molecule. Particularly suitable zwitterionic surfactants are the so-called betaines such as the N-alkyl-N,N-dimethylammonium glycinates, for example the cocoalkyldimethyl-ammonium glycinate, N-acylaminopropyl-N,N-dimethylammonium glycinates, for example the cocoacylaminopropyldimethylammonium glycinate; and 2-alkyl-3-carboxylmethyl-3-hydroxyethylimidazolines each having 8 to 18 C atoms in the alkyl or acyl group, and the cocoacylaminoethylhydroxyethylcarboxymethyl glycinate. A particularly preferred fatty amide derivative is that known under the CTFA name cocamidopropyl betaine.

Emulsifiers which are likewise suitable are ampholytic surfactants. Ampholytic surfactants means surface-active compounds which, apart from a C_{8,18}-alkyl or -acyl group, comprise at least one free amino group and at least one —COOH or —S0₃H group in the molecule and are able to form inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 18 C atoms in the alkyl group.

Particularly preferred ampholytic surfactants are N-cocoalkylaminopropionate, cocoacylaminoethylaminopropionate and C_{12/18}-acylsarcosine. Besides the ampholytic emulsifiers, also suitable are quarternary emulsifiers, with particular preference for those of the ester quat type, preferably methyl-quaternized di-fatty acid triethanolamine ester salts. Anionic emulsifiers which can also be employed are alkyl ether sulfates, monoglyceride sulfates, fatty acid sulfates, sulfosuccinates and/or ether carboxylic acids.

Suitable oily substances are guerbet alcohols based on fatty alcohols having 6 to 18, preferably 8 to 10, carbon atoms, esters of linear C₆-C₂₂ fatty acids with linear C₆-C₂₂ fatty alcohols, esters of branched C₆-C₁₃ carboxylic acids with linear C₆-C₂₂ fatty alcohols, esters of linear C₆-C₂₂ fatty acids with branched alcohols, especially 2-ethylhexanol, esters of linear and/or branched fatty acids with polyhydric alcohols (such as, for example, propylene glycol, dimerdiol or trimertriol) and/or guerbet alcohols, triglycerides based on C₆-C₁₀ fatty acids, liquid mono/di-, triglyceride mixtures based on C₆-C₁₈ fatty acids, esters of C₆-C₂₂ fatty alcohols and/or guerbet alcohols with aromatic carboxylic acids, especially benzoic acid, esters of C₂-C₁₂ dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂ fatty alcohol carbonates, guerbet carbonates, esters of benzoic acid with linear and/or branched C₆-C₂₂ alcohols (e.g. Finsolv® TN), dialkyl ethers, ring-opened products of epoxidized fatty acid esters with polyols, silicone oils and/or aliphatic or naphthenic hydrocarbons. Further oily substances which can be employed are silicone compounds, for example dimethylpolysiloxanes, methylphenylpolysiloxanes, cyclic silicones and amino-, fatty acid-, alcohol-, polyether-, epoxy-, fluorine-, alkyl- and/or glycoside-modified silicone compounds which may at room temperature be both in liquid form and in the form of a resin. The oily substances may be present in the compositions of the invention in amounts of from 1 to 90, preferably 5 to 80, and in particular 10 to 50, % by weight based on the composition.

The duration of action on the skin can be signified prolonged by coupling appropriate compounds to a keratin-binding polypeptide (i). The coupling takes place as described above, and formulation and application take place by methods known to the skilled worker. Effector molecules (ii) suitable in particular for deodorants are: perfume oils, cyclodextrines, ion exchangers, zinc ricinoleate, antimicrobial/bacteriostatic compounds (e.g. DCMX, Irgasan DP 300, TCC).

Suitable for antipersipirants are: tannins, and zinc/aluminum salts.

A further area of application of the substances of the invention is the therapeutic or prophylactic use for certain disorders of the skin and of the mucous membranes. It is advantageous, especially in the oral, pharyngeal and nasal spaces, for active substances for therapy/prophylaxis to be bound more strongly and for a longer time via a keratin-binding domain. Areas of application thereof are, in particular:
viral diseases (e.g. herpes, coxsackie, varicella zoster, cytomegalovirus etc)
bacterial diseases (e.g. TB, syphilis etc.)
fungal diseases (e.g. candida, cryptococcus, histoplasmosis, aspergillus, mucormycosis etc.)
neoplastic diseases (e.g. melanomas, adenomas etc.)
autoimmune diseases (e.g. PEMPHIGUS VULGARIS, BULLOUS PEMPHIGOID, SYSTEMIC LUPUS ERYTHEMATOSIS etc.)
sunburn
parasitic infestation (e.g. ticks, mites, fleas etc.)
insect contact (e.g. blood-sucking insects such as anopheles etc.)

The substances suitable for therapy or prophylaxis (e.g. corticoids, immunosuppressant compounds, antibiotics, antimycotics, antiviral compounds, insect repellent etc.) can be coupled via the linkers described above (a linker to be optimized according to the functionality to be coupled) to the keratin-binding polypeptides (i).

### EXAMPLE 1

### Expression Vectors and Production Strains

Various expression vectors were tested for the expression of the keratin-binding domains (KBD). For this, various promoters were used (e.g. IPTG-inducible, rhamnose-inducible, arabinose-inducible, methanol-inducible, constitutive promoters, etc.). Constructs were likewise tested in which the KBD were expressed as fusion proteins (e.g. as fusion with thioredoxin, or eGFP, or YaaD [B. subtilis, SWISS-PROT: P37527, PDX1], etc.). Here, both the described KBD-B (keratin-binding domain B), and KBD-C (keratin-binding domain C), and the combination of the two domains KBD-BC were expressed using the various expression systems. The vector constructs mentioned are nonlimiting for the claim.

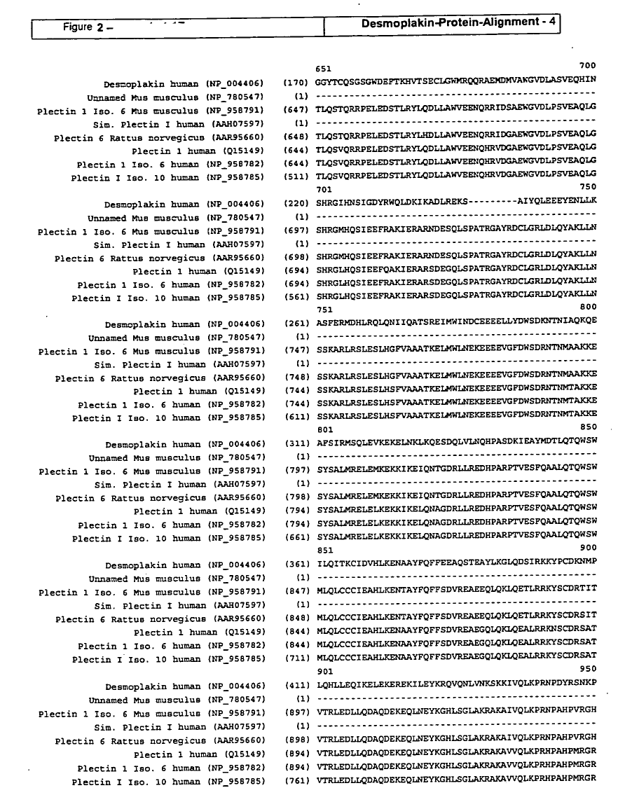

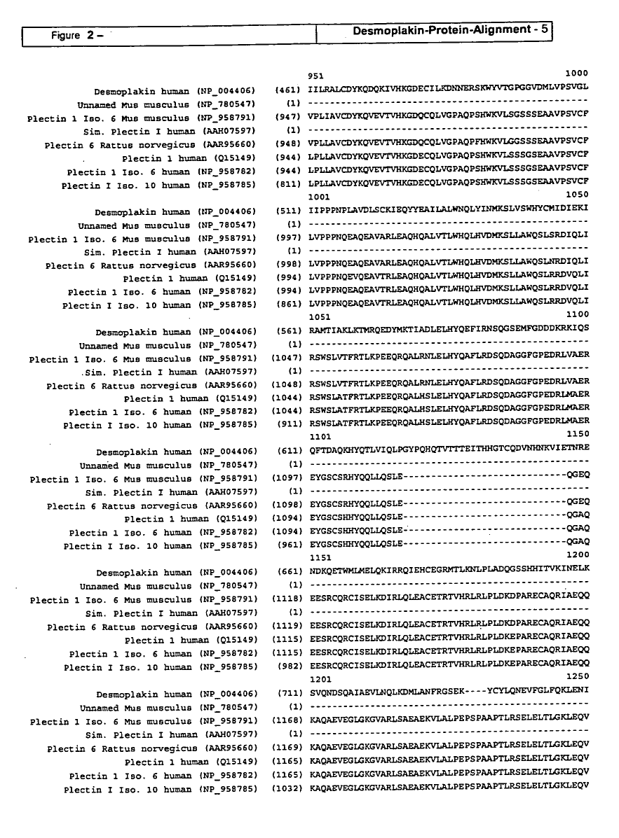

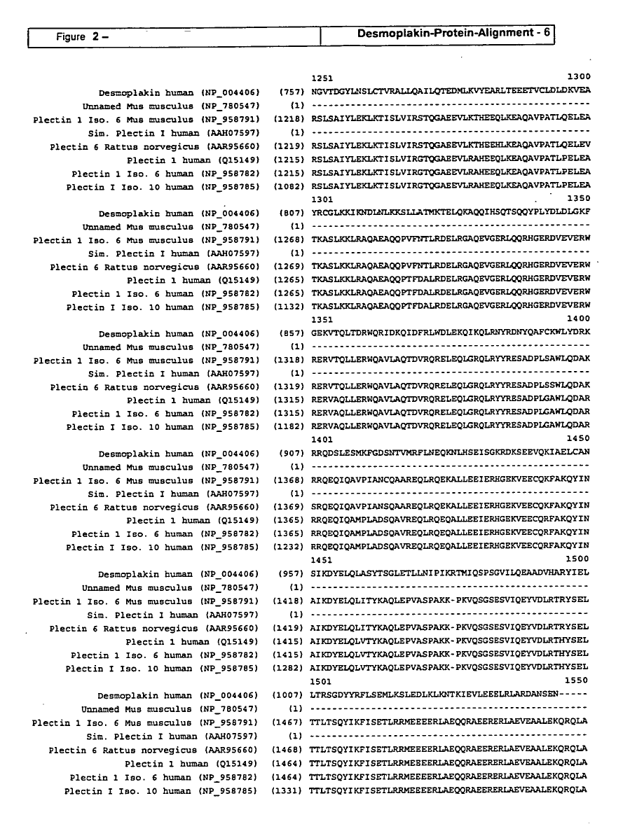

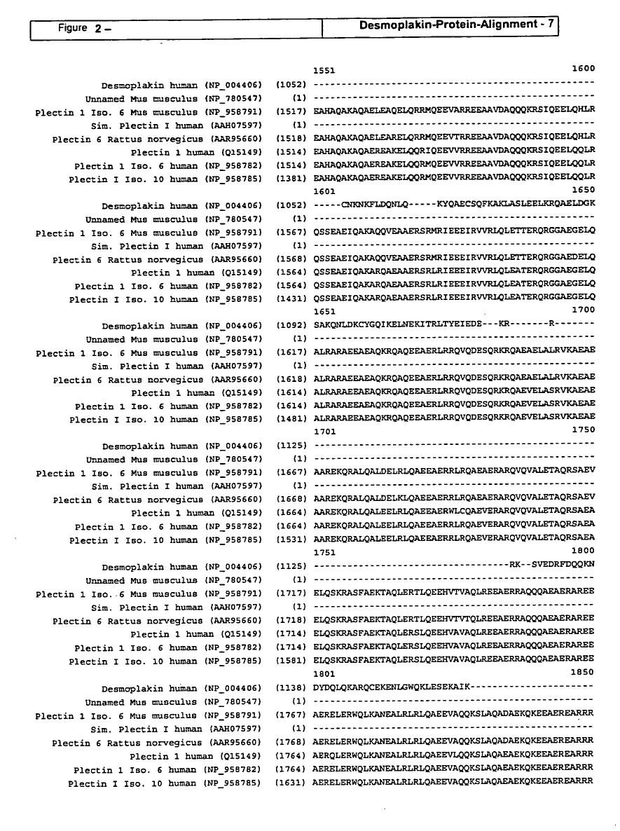

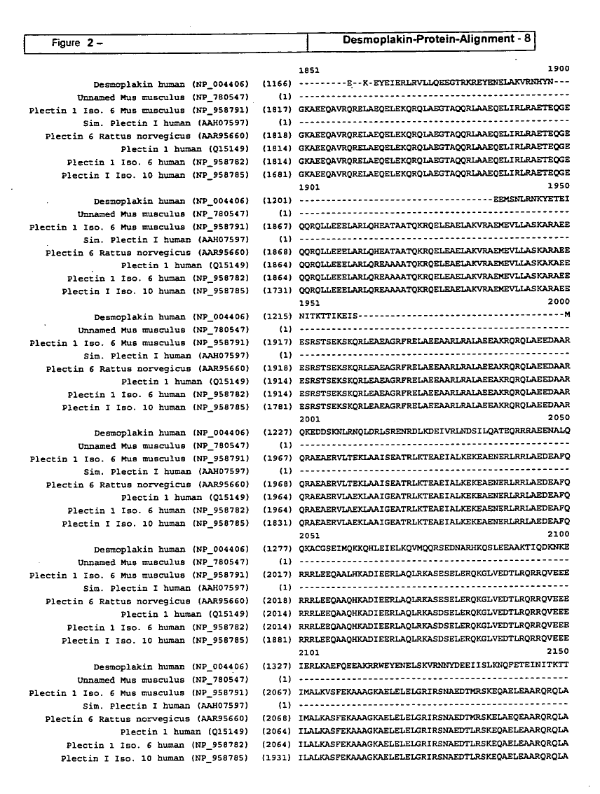

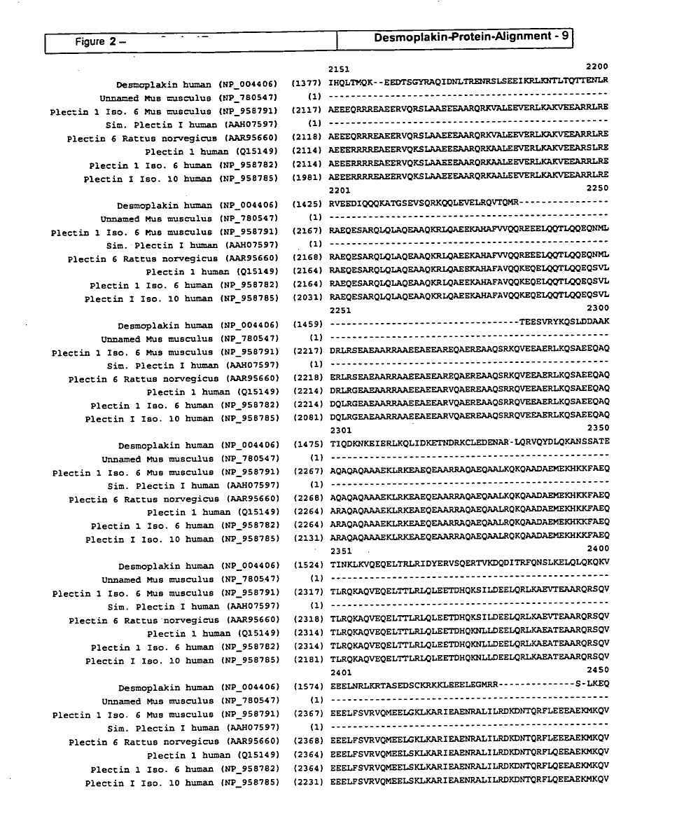

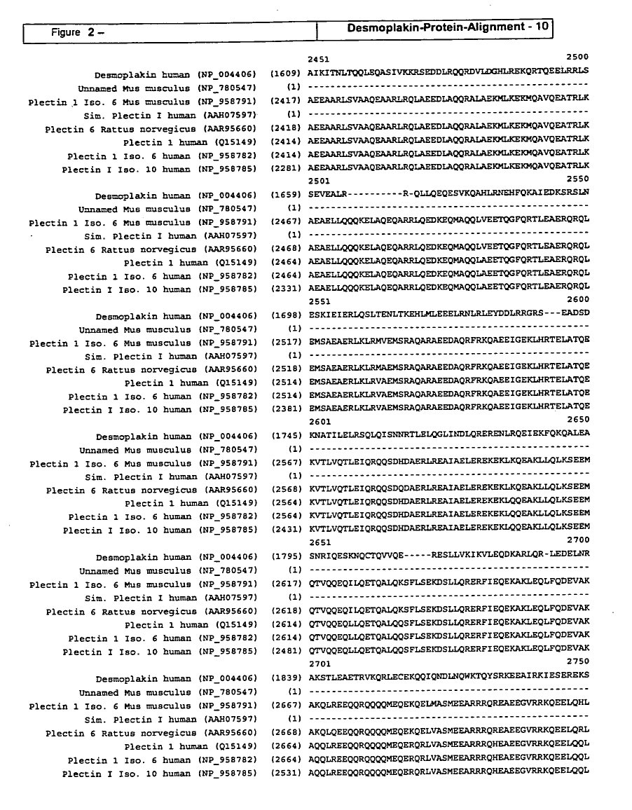

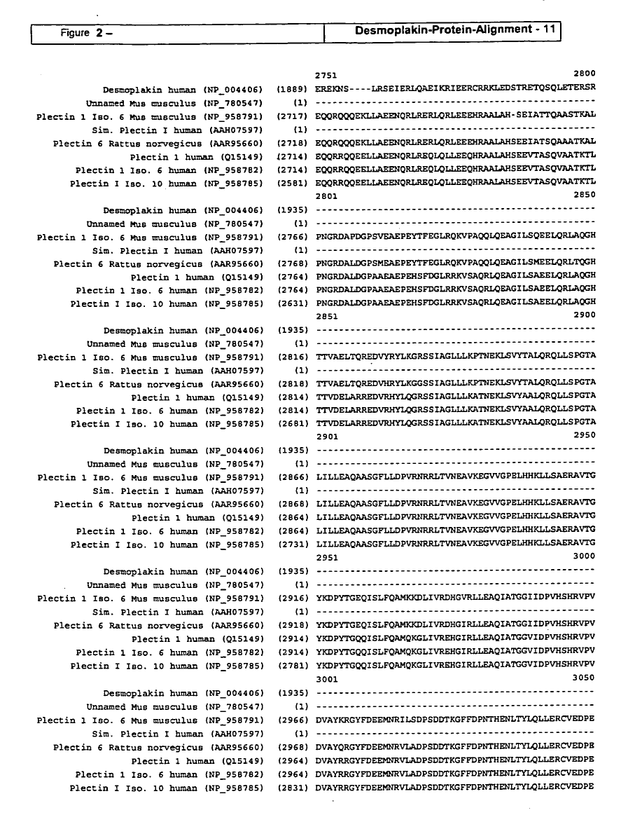

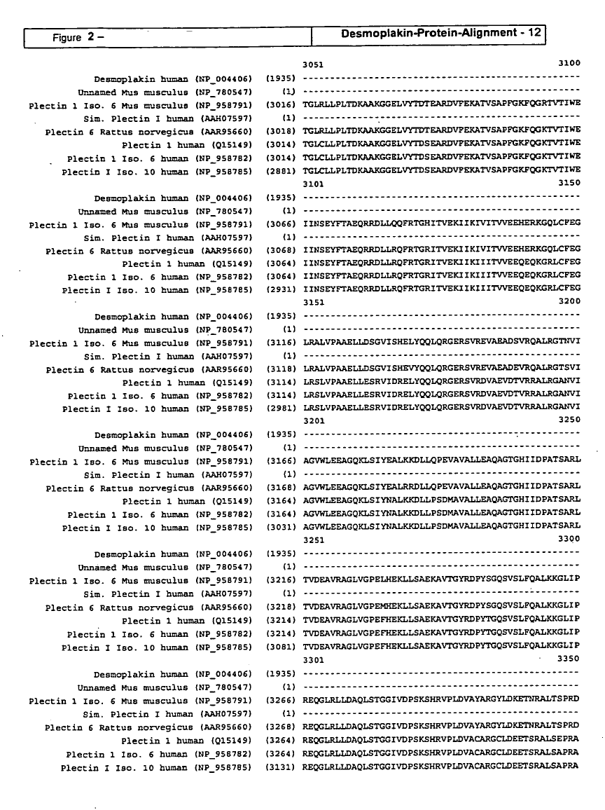

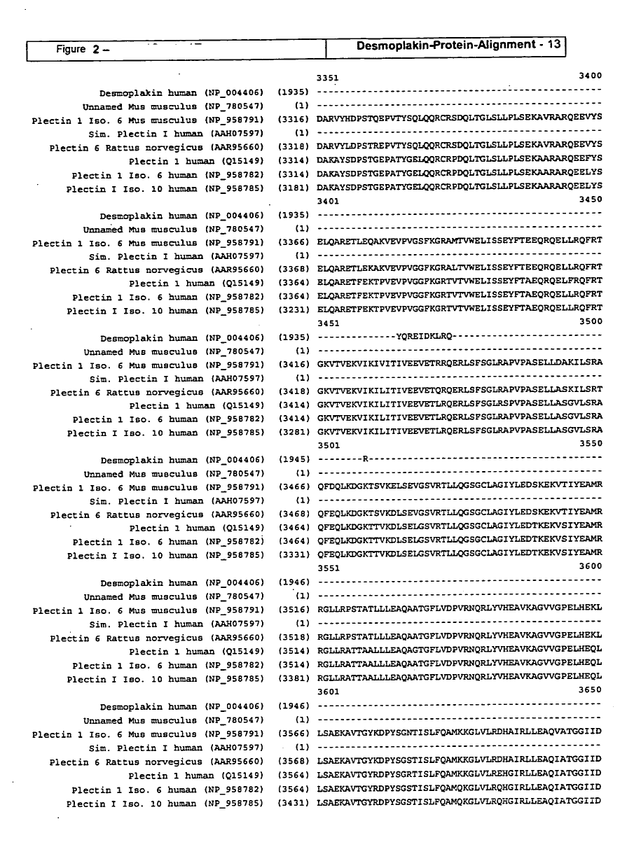

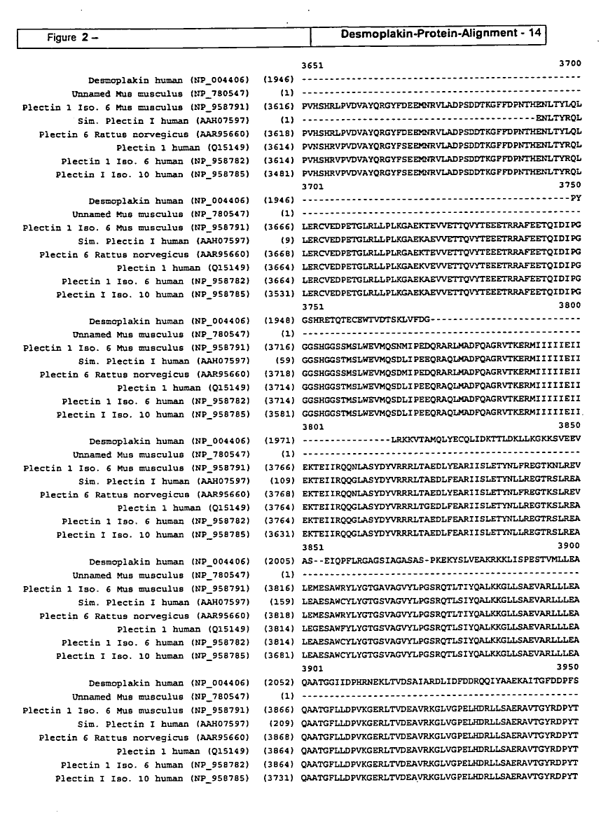

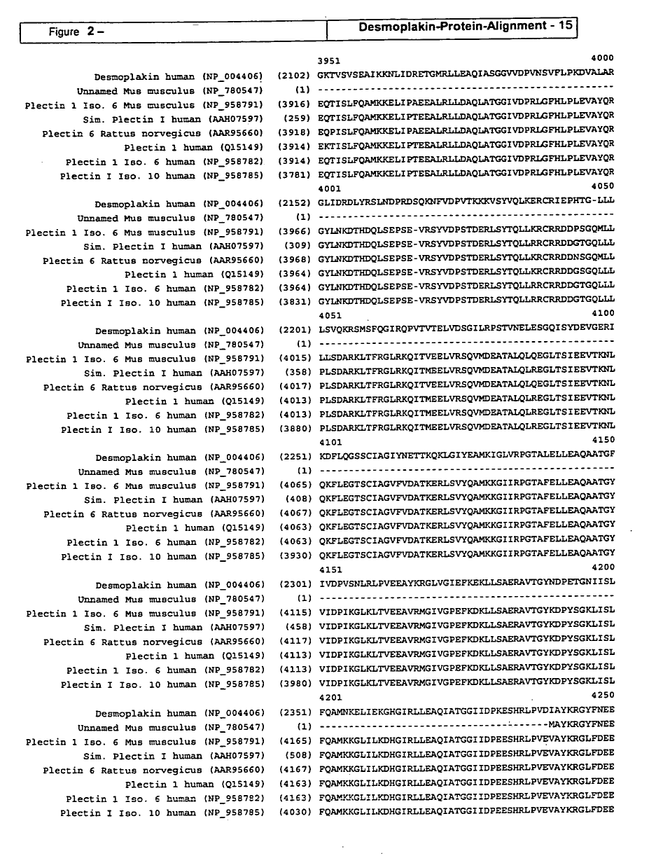

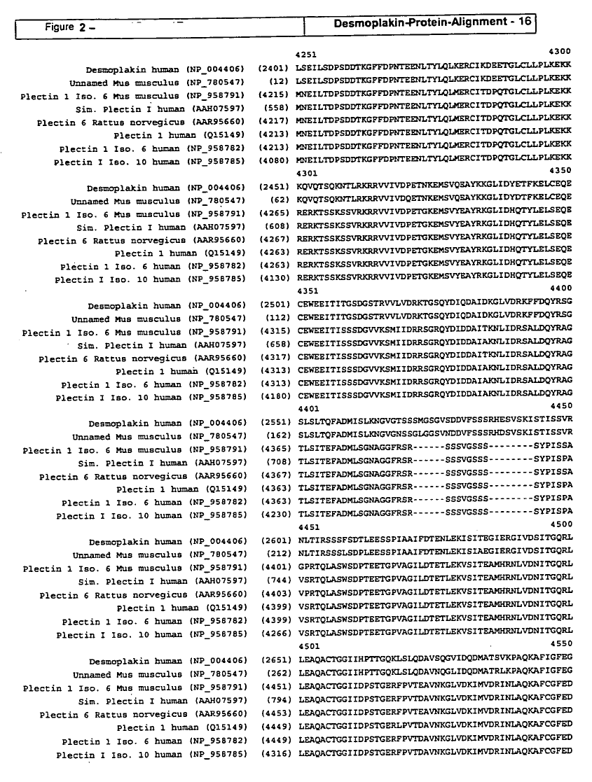

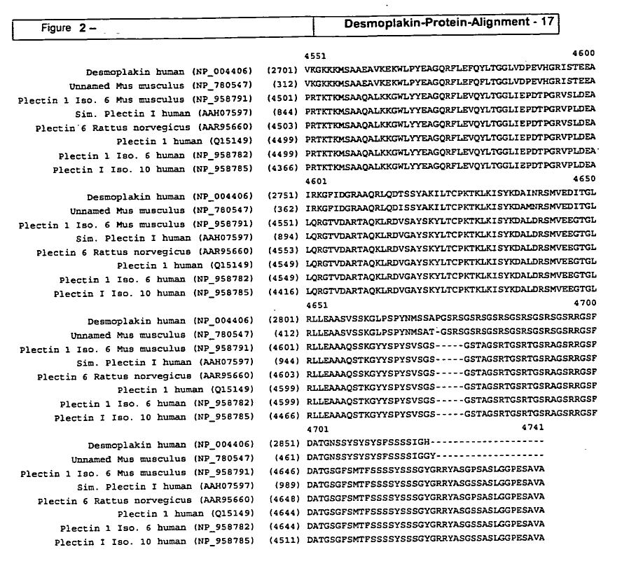

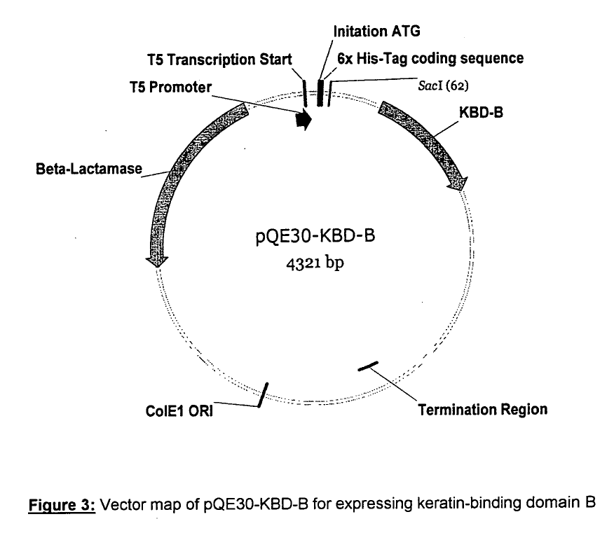

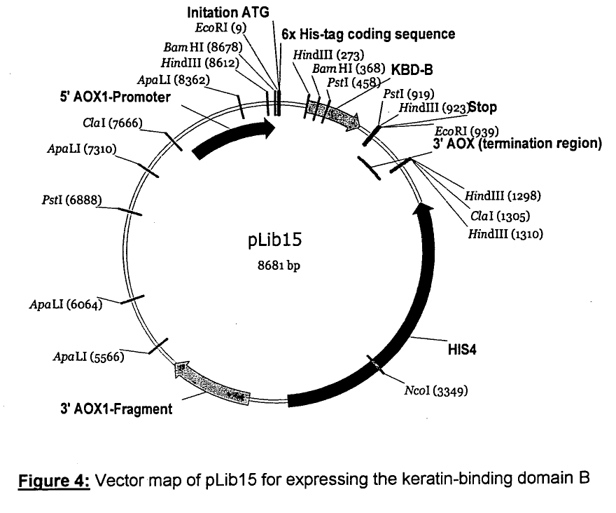

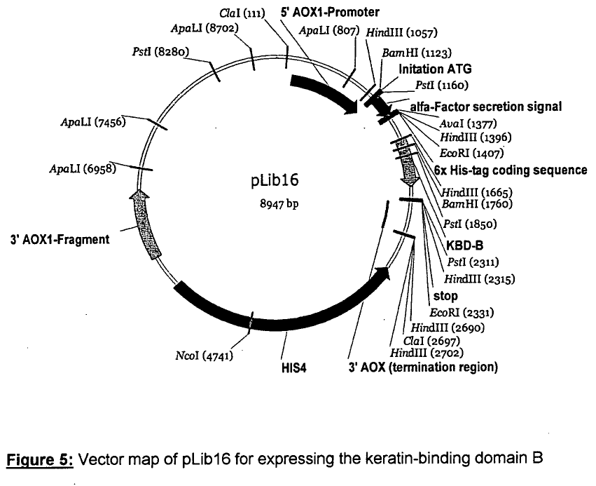

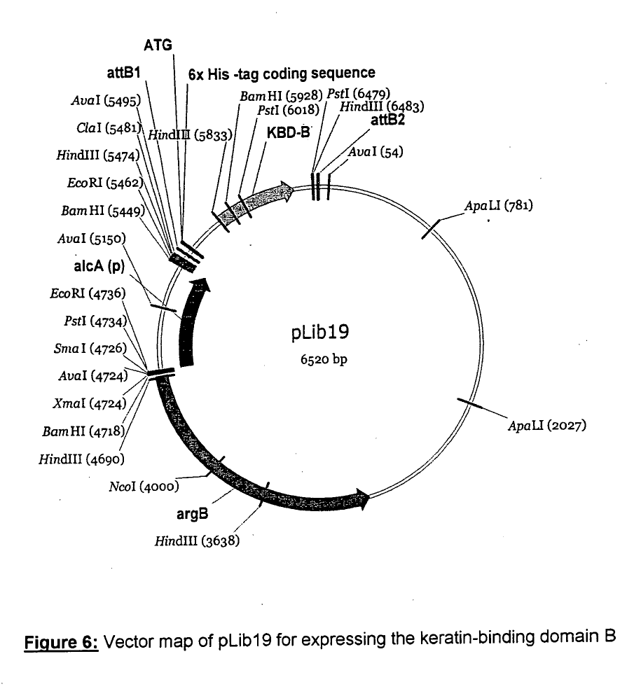

Given by way of representative as an example is the vector map of the IPTG-inducible vector pQE30-KBD-B (FIG. 3), of the methanol-inducible vectors pLib15 (FIG. 4) and pLib16 (FIG. 5), and of the inducible vector pLib19 (FIG. 6). The procedure for KBD-C may also be analogous to the described vector constructions and expressions.

For the expression of the KBD, various production hosts were used, such as, for example, E. coli strains (see Ex. 2; e.g. XL10-Gold [Stratagene], BL21-CodonPlus [Stratagene], and others). However, other bacterial production hosts, such as, for example, Bacillus megaterium or Bacillus subtilis, were also used. In the case of the KBD expression in B. megaterium, the procedure was carried out analogously to: Barg, H., Malten, M. & Jahn, D. (2005). Protein and vitamin production in Bacillus megaterium. In Methods in Biotechnology-Microbial Products and Biotransformations (Barredo, J.-L., ed.).

The fungal production strains used were Pichia pastoris (see Ex. 3; e.g. GS115 and KM71 [both from Invitrogen]; and others) and Aspergillus nidulans (see Ex. 4; e.g. RMS011 [Stringer, M A, Dean, R A, Sewall, T C, Timberlake, W E (1991) Rodletless, a new Aspergillus developmental mutant induced by direct gene activation. Genes Dev 5:1161-1171] und SRF200 [Karos, M, Fischer, R (1999) Molecular characterization of HymA, an evolutionarily highly conserved and highly expressed protein of Aspergillus nidulans. Mol Genet Genomics 260:510-521], and others). However, it is also possible to use other fungal production hosts, such as, for example, Aspergillus niger (KBD expression analogous to EP 0635574A1 and/or WO 98/46772) for the KBD expression.

### EXAMPLE 2

### KBD Expression in E. coli Strains with IPTG Inducible Promoters, e.g. by the Expression Plasmid pQE30-KBD-B

For the expression, various production hosts were used, such as, for example, various E. coli strains (e.g. XL10-Gold [Stratagene], BL21-CodonPlus [Stratagene], and others), Bacillus megaterium, Bacillus subtilis etc.

Described here—by way of representation as an example—is the cloning and expression of KBD-B by E. coli, transformed with pQE30-KBD-B:

### Cloning of pQE30-KBD-B

Lambda-MaxiDNA (DNA-Lambda Maxi Kit, Qiagen) was prepared from a cDNA bank of human keratinocytes (BD Bioscience, Clontech, Human Keratinocyte cDNA, foreskin, primary culture in log phase, vector: λgt11).

The PCR was carried out using the following oligonucleotides:

Bag 43
(5′- GGTCAGTTACGTGCAGCTGAAGG -3′)
and
Bag 44
(5′- GCTGAGGCTGCCGGATCG -3′)

The resulting PCR product about 1102 bp in size was cut out of an agarose gel and purified.
Using the purified PCR product as template, a 2nd PCR was then carried out:

Oligonucleotides used:

Bag 53:
(5′- CGCGCCTCGAGCCACATACTGGTCTGC -3′)
and
Bag 51
(5′- GCTTAGCTGAGGCTGCCGGATCG -3′)

The resulting PCR product about 1073 bp in size was cut out of an agarose gel, purified and cloned in the following vector: pCR2.1-TOPO (Invitrogen).
The resulting vector pCR2.1-TOPO+KBD-B (5027 bp) was then transformed, amplified in E. coli, then cleaved with XhoI and EcoRI and the resulting KBD-B fragment was cloned in pBAD/HisA (Invitrogen; likewise cleaved with XhoI and EcoRI).
The newly formed vector pBAD/HisA+KBD-B (5171 bp) was again cleaved with SacI and StuI and the resulting KBD-B fragment was cloned in pQE30 (Qiagen; cleaved with SacI and SmaI). The resulting expression vector pQE30-KBD-B (4321 bp; see also FIG. 3) was used for the following KBD-B expressions.

The KBD-B expressed by the vector pQE30-KBD-B in E. coli additionally included, on the N-terminus, besides the polypeptide sequence SEQ ID NO: 1 position 2193-2481, the amino acids MRGSHHHHHHGSACEL, and, on the C-terminus, the amino acids GVDLQPSLIS.

Expression of KBD-B by pQE30-KBD-B in E. coli
Precultures were inoculated from plate or glycerol culture with pQE30-KBD-B transformed E. coli strains (e.g. XL10-Gold [Stratagene]). Depending on the size of the main culture, inoculation with LB medium (about 1:100) was carried out in a tube or a small flask.
Antibiotics were used according to the strain used (for pQE30-KBD-B ampicillin 100 μg/ml).
Incubation was carried out at 250 rpm and 37° C.
The main culture was inoculated about 1:100 with preculture, main culture: LB medium or suitable minimal medium with the respective antibiotics. Incubation at 250 rpm and 37° C.
Induction was carried out with 1 mM IPTG above an OD (600 nm) of 0.5.
After induction for 4 h, the cells were centrifuged off.

In fermenters the procedure was analogous, although it was possible to carry out induction at much higher OD units and thus to considerably increase the cell and protein yield.

### EXAMPLE 3

### Intracellular and Secretory Expression of KBD by Means of Pichia pastoris Strains Using Methanol-Inducible Promoters, e.g. Through the Expression Plasmids pLib 15 and pLib 16 (shaking flask)

For the KBD expression, various Pichia pastoris strains were used, such as, for example, GS115 and KM71 (Pichia Expression Kit, Version M; Invitrogen Life Technologies).

Described here is—by way of representative as an example—the expression of KBD-B by P. pastoris, transformed with pLib15 (intracellular expression, vector see FIG. 4) or pLib16 (secretory expression, vector see FIG. 5).
For the construction of pLib15, a KBD-B-encoding DNA fragment about 930 bp in size was amplified by means of PCR using the oligonucleotides Lib148 (5′-GCTAAGGAATTCACCATGCATCACCATCACCATCACGAGCCACATACTGGTC TGCT-3′) and Lib149 (5′-GCTGGAGAAMTTCTCAGCTAATTAAGCTTGGCTGCA-3′), and the vector pQE30-KBD-B (Example 2, FIG. 3) as templates. Here, EcoRI restriction sites were introduced at both ends of the PCR products.
For the construction of pLib16, a KBD-B-encoding DNA fragment about 930 bp in size was amplified by means of PCR using the oligonucleotides Lib149 (5′-GCTGGAGAATTCTCAGCTAATTAAGCTTGGCTGCA-3′) and Lib150 (5′-GCTAAGGAATTCCATCACCATCACCATCACGAGCCACATACTGGTCTGCT-3′) and the vector pQE30-KBD-B (Example 2, FIG. 3) as templates. Here, EcoRI restriction sites were introduced at both ends of the PCR products.
The PCR product which was amplified with the oligonucleotides Lib148/Lib149 was digested with EcoRI and ligated into the EcoRI-cleaved vector pPIC3.5 (Pichia Expression Kit, Version M, Invitrogen). The correct KBD-B amplification was checked by sequencing the vector pLib15 (FIG. 4) resulting from the ligation.
The PCR product which was amplified with the oligonucleotides Lib149/Lib150 was digested with EcoRI and ligated into the EcoRI-cleaved vector pPIC9 (Pichia Expression Kit, Version M, Invitrogen). The correct KBD-B amplification was checked by sequencing the vector pLib16 (FIG. 5) resulting from the ligation.
Electrocompetent cells and spheroplasts of the P. pastoris strains were transformed with the circular and StuI-linearized vectors pLib15 and pLib16
The transformants were analyzed by means of PCR and Southern blotting using chromosomal DNA.
For the preculture, KBD-B-expressing P. pastoris transformants were inoculated from plate or glycerol culture. Depending on the size of the main culture, inoculation with MGY, BMG or BMGY medium (Pichia-Expression-Kit, Version M, Invitrogen) (about 1:100) was carried out in a tube or a small flask.
The culture was incubated at 250-300 rpm and 30° C. until OD₆₀₀=2-6.
The cells were harvested with 1500-3000×g for 5 min at room temperature.
For the main culture, the harvested cell pellet was taken up at an OD₆₀₀=1 in methanol-comprising mM, BMM or BMMY medium (Pichia-Expression-Kit, Version M, Invitrogen) in order to induce the expression.
The main culture was incubated at 250-300 rpm and 30° C. for 1-96 h.
The induction was maintained every 24 h by adding 100% methanol at a methanol end concentration of 0.5%.
In the case of intracellular expression, the harvesting and disruption of the cells was carried out after the end of the main culture by means of a Menton-Gaulin.
In the case of secretory expression, the culture supernatant was collected and the KBD-B was purified from it directly.
The KBD-B expressed intracellularly in P. pastoris (pLib15) included, besides the polypeptide sequence SEQ ID NO: 1 position 2193-2481, additionally, at the N-terminus, the amino acids MHHHHHH, and, at the C-terminus, the amino acids GVDLQPSLIS.
The KBD-B expressed secretorily in P. pastoris (pLib16) included, prior to processing, besides the polypeptide sequence SEQ ID NO: 1 position 2193-2481, additionally at the N-terminus the amino acids MRFPSIFTAVLFMSSALAAPVNTTTEDETAQIPAEAVIGYSDLEGDFDVAVLPFS NSTNNGLLFINTTIASIAAKEEGVSLEKREAEAYVEFHHHHHH, and, at the C-terminus, the amino acids GVDLQPSLIS.
The KBD-B processed and secreted by means of P. pastoris (pLib16) included, besides the polypeptide sequence SEQ ID NO: 1 position 2193-2481, additionally at the N-terminus the amino acids YVEFHHHHHH, and at the C-terminus the amino acids GVDLQPSLIS.

### EXAMPLE 4

### Expression of KBD by Means of Aspergillus nidulans Strains Using the Inducible alcA Promoter, e.g. Through the Expression Plasmid pLib 19 (Shaking Flask)

For the expression, A. nidulans wild type strains were used, such as, for example, RMS011 or SRF200. Described here is—by way of representation as an example—the expression of KBD-B by A. nidulans, transformed with pLib19 (FIG. 6).
For the construction of pLib19, a KBD-B-encoding DNA fragment about 900 bp in size was amplified by means of PCR using the oligonucleotides Lib151 (5′-CACCATGCATCACCATCACCATCACGAGCCACATACTGGTCTGCT-3′) and Lib152 (5-GCTAATTAAGCTTGGCTGCA-3′), and the vector pQE30-KBD-B (Example 2, FIG. 3) as template. The PCR product was ligated into the vector pENTR/D (pENTR™ Directional TOPO® Cloning Kit, Version E, Invitrogen). The correct KBD-B amplification was checked by sequencing.
The recombination of the KBD-B encoding DNA fragment was carried out into the vector pMT-OvE (Toews M W, Warmbold J, Konzack S, Rischitor P, Veith D, Vienken K, Vinuesa C, Wei H, Fischer R; Establishment of mRFP1 as a fluorescent marker in Aspergillus nidulans and construction of expression vectors for high-throughput protein tagging using recombination in vitro (GATEWAY). (2004) Curr Genet 45: 383-389) using the “Gateway® LR Clonase™ enzyme mix” (Invitrogen). This produced the vector pLib19 (FIG. 6).
Protoplasts of the A. nidulans wild type strains were transformed with the circular vector pLib19. The transformants were analyzed by means of PCR and Southern blotting using chromosomal DNA.
For the preculture of KBD-B-expressing A. nidulans transformants, 100 ml of minimal medium (0.6% NaNO₃; 0.152% KH₂PO₄; 0.052% KCl [pH 6.5]; 0.8% glucose; 0.05% MgSO₄; 1 ml trace element solution [1 g/l FeSO₄×₇H₂O; 8.8 g/l ZnSO₄×₇H₂O; 0.4 g/l CuSO₄×₅H₂O; 0.15 g/l MnSO₄×₄H₂O; 0.1 g/l Na₂B₄O₇×10H₂O; 0.05 g/l (NH₄)₆Mo₇O₂₄×₄H₂O], +strain-specific supplements) or 100 ml of complete medium (2% malt extract; 0.1% peptone; 2% glucose; +strain-specific supplements) were inoculated in 500 ml flasks with 10⁶-10⁷ spores and incubated for 16-24 h at 200-250 rpm and 37° C.
After the preculture, the fungal mycelium was harvested by filtration, washed with distilled water and transferred to flasks with 100-500 ml of fresh minimal medium. In this main culture medium, 0.1% fructose was used instead of glucose as the C-source. To induce the KBD expression, ethanol (1% final concentration) or glycerol (50 mM) or sodium acetate (50 mM) or ethylamine or threonine were additionally added to the medium. The additives mentioned for inducing the expression are not limiting for the claim. The main culture was incubated for a further 5-48 h at 200-250 rpm and 37° C.
After the end of the culture, the fungal mycelium was harvested with 1500-3000×g for 5 min at room temperature and disrupted by means of a Menton-Gaulin.
Besides the polypeptide sequence SEQ ID NO: 1 position 2193-2481, the KBD-B expressed in A. nidulans (pLib19) additionally included, at the N-terminus, the amino acids MHHHHHH, and, at the C-terminus, the amino acids KGGRADPAFLYKWMIRLLTKPERKLLEGGPGTQLLFPLVRVNCALGVIMVIAVS CVKLLSAHNSTQHTSRKHKV.

### EXAMPLE 5

### Cell Disruption and Inclusion Body Purification

### pQE30-KBD-B

Solubly expressed KBD could be used directly following purification. Insolubly expressed KBD (e.g. in inclusion bodies) was purified as follows:
The fermenter was centrifuged, the pellet was suspended in 20 mM phosphate buffer pH=7.4 and disrupted by means of a Menton-Gaulin.
The disrupted cells were centrifuged again (15 000 g), the pellet from this was treated with 20 mM phosphate, 500 mM NaCl and 8 M urea and so stirred. (Dissolution of the Inclusion Bodies)
The pH of the supernatant was adjusted to 7.5.
Centrifugation was then carried out again and the supernatant was applied to an Ni chelate Sepharose column.

### EXAMPLE 6

### Purification of Keratin-Binding Domain B on Ni Chelate Sepharose

The KBD could be purified chromatographically through the attached His tag over an Ni column.

Column material: Ni-Sepharose High Performance
Amersham Biosciences order No.: 17-5268-02

The material was packed into a column (e.g. diameter 2.6 cm, height 10 cm) and equilibrated with buffer A+4% buffer B (corresponds to 20 mM imidazole).

The protein extract (see e.g. cell disruption and inclusion body purification) was applied to the column at pH 7.5 using a Superloop (ÄKTA system) (flow about 5 ml/min).

Following application, washing was carried out with buffer A+20 mM imidazole.

Elution was carried out with buffer B (500 mM imidazole in buffer A).

The eluate was collected in fractions using a fraction collector.

### Buffer A: 20 mM sodium dihydrogenphosphate

500 mM NaCl
8 M urea
pH=7.40

### Buffer B: 20 mM sodium dihydrogenphosphate

500 mM NaCl
8 M urea
500 mM imidazole
pH=7.40

### EXAMPLE 7

### Renaturation of Keratin-Binding Domain B

Insolubly expressed keratin-binding domain (e.g. from inclusion bodies) can be renatured and thus activated as follows:

### Method 1: Discontinuous Dialysis

6.5 ml of Cellytic IB (Sigma, order No. C5236) and 5 mM DTT were added to 6.5 ml of KBD-B inclusion bodies in 8 M urea (Ni chelate eluate, HiTrap). The solution to be renatured was then poured into a dialysis tube (Spectrum: Spectra Por MWCO:12-14 kD).

Carry out dialysis for about 12 hours against 1 L of 6 M urea solution at 4° C. with careful stirring.

500 ml of 25 mM Tris/HCl pH=7.50 were added and dialysis was carried out like this for 9 hours at 4° C. Subsequent addition of a further 250 ml of the Tris buffer (see above) and dialysis for a further 12 hours.

500 ml of 25 mM Tris/HCl pH=7.50 were then added again and dialysis was carried out like this for 9 hours at 4° C. Subsequent addition of a further 250 ml of the Tris buffer (see above) and dialysis for a further 12 hours.

500 ml of 25 mM Tris/HCl pH=7.50 were then added again and dialysis was carried out like this for 9 hours at 4° C. The dialysis tube containing the dialyzate was then placed into 2L: 25 mM Tris+150 mM NaCl pH=7.50. Dialysis was then carried out again at 4° C. for 12 hours.

The contents of the dialysis tube were then removed.

### Method 2: Continuous Dialysis

20 ml of KBD-B inclusion bodies in 8 M urea (Ni chelate eluate, HiTrap) were treated with 10 ml of Cellytic IB (Sigma, order No. C5236) and 5 mM DTT. The solution was then poured into a dialysis chamber: Slide-A-Lyzer Dialyses Cassette PIERCE, MWCO: 10 kD. Order No.: 66830.

Dialysis was then carried out for about 1 hour against 1 L 6 M urea solution at 4° C.

Then, over a period of 48 h, 2 l of the following buffer were metered in continuously by means of a peristaltic pump: 25 mM Tris/HCl pH=7.5.

The dialysis tube containing the dialyzate was then added to 2 l of the end buffer:

25 mM Tris+150 mM NaCl pH=7.50 and dialysis was carried out for about 12 hours at 4° C.

The contents of the dialysis tube were then removed.

### EXAMPLE 8

### Binding to Skin 1 (Qualitative)

A visual qualitative test was developed in order to examine whether KBD binds to skin.

### Solutions Used:

Blocking solution: DIG Wash+Buffer set 1585762 Boehringer MA (10× solution) diluted in TBS.

### TBS: 20 mM Tris; 150 mM NaCl pH 7.5

### TTBS: TBS+0.05% Tween20

The first step is the transfer of the outer keratin layer of the skin to a stable support. For this purpose, a transparent adhesive tape is firmly applied to depilated human skin and removed again. The test can be carried out directly on the transparent adhesive strip, or the adhering keratin layer can be transferred to a glass slide through renewed adhesion. Binding was demonstrated as follows:
For incubation with the various reagents, transfer to a Falcon vessel
If appropriate addition of ethanol for degreasing, removal of ethanol and drying of the slide
Incubation with blocking buffer for 1 h at room temperature
2× washing for 5 min with TTBS
1× washing for 5 min with TBS
Incubation with the KBD to be tested (coupled to tag—e.g. His₆, HA etc.) or control protein in TBS/0.05% Tween 20 for 2-4 h at room temperature
Removal of the supernatant
3× washing with TBS
Incubation for 1 h at room temperature with monoclonal anti-polyhistidine (or specific KBD rabbit) antibodies, diluted 1:2000 in TBS+0.01% blocking
2× washing for 5 min with TTBS
1× washing for 5 min with TBS
Incubation for 1 h at room temperature with anti-mouse IgG alkaline-phosphatase conjugate, diluted 1:5000 in TBS+0.01% blocking
2× washing for 5 min with TTBS
1× washing for 5 min with TBS
Addition of phosphatase substrate (NBT-BCIP; Boehringer MA 1 tablet/40 ml of water 2.5 min; stop: with water)
Optical detection of the colored precipitate with the naked eye or using a microscope. A blue colored precipitate indicates that KBD has bound to the skin.

### EXAMPLE 9

### Binding to Skin 2 (Quantitative)

A quantitative test was developed with which the hair/skin binding strength of the KBD can be compared with nonspecific proteins.

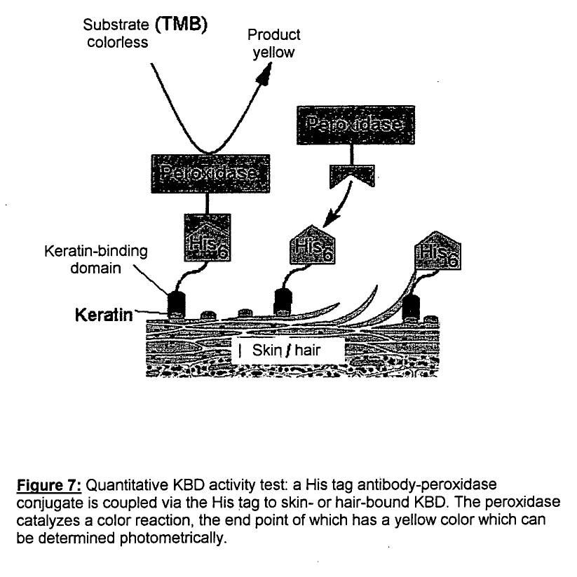

A 5 mm cork borer was used to bore a section out of a thawed dry piece of skin without hair (human or pig) (or in the case of a surface test a section of skin is inserted into a Falcon lid). The sample of skin was then brought to a thickness of 2-3 mm in order to remove any tissue present. The skin sample was then transferred to an Eppendorf vessel (protein low-bind) in order to carry out the binding demonstration (see also FIG. 7):
2× washing with PBS/0.05% Tween 20
Addition of 1 ml of 1% BSA in PBS and incubation for 1 h at room temperature, gentle swirling movements (900 rpm).
Removal of the supernatant
Addition of 100 μg of KBD in PBS with 0.05% Tween 20; incubation for 2 h at room temperature and gentle swimming movements (900 rpm).
Removal of the supernatant
3× washing with PBS/0.05% Tween 20
Incubation with 1 ml of monoclonal mouse anti-tag (His6 or HA or specific KBD) antibodies with peroxidase conjugate (1:2000 in PBS with 0.05% Tween 20) [Monoclonal AntipolyHistidine Peroxidase Conjugate, produced in mouse, lyophilized powder, Sigma] for 2-4 h at room temperature, gentle swirling movement (900 rpm)
3× washing with PBS/0.05% Tween 20
Addition of peroxidase substrate (1 ml/Eppendorf vessel; composition see below)
Allow reaction to run until a blue coloration (about 90 seconds).
Stop the reaction with 100 μl of 2 μM H₂SO₄.
The absorption was measured at 405 nm.

### Peroxidase Substrate (Prepare Shortly Beforehand):

0.1 ml TMB solution (42 mM TMB in DMSO)
+10 ml substrate buffer (0.1 M sodium acetate pH 4.9)
+14.7 μl H₂O₂ 3% strength

### EXAMPLE 10

### Binding to Hair (Quantitative)

In order to be able to demonstrate the binding strength of KBD to hair also relative to other proteins, a quantitative assay was developed (see also FIG. 7). In this test, hair was firstly incubated with KBD and excess KBD was washed off. An antibody-peroxidase conjugate was then coupled via the His tag of the KBD. Nonbound antibody-peroxidase conjugate was washed off again. The bound antibody-peroxidase conjugate [Monoclonal AntipolyHistidine Peroxidase Conjugate, produced in mouse, lyophilized powder, Sigma] can convert a colorless substrate (TMB) into a colored product, which can be measured photometrically at 405 nm. The intensity of the absorption indicates the amount of bound KBD or comparison protein. The comparison protein chosen was, for example, YaaD from B. subtilis, which likewise had—as is necessary for this test—a His tag for the detection. Instead of the His tag, other specific antibodies conjugated with peroxidase can also be used.

5 mg of hair (human) are cut into sections 5 mm in length and transferred to Eppendorf vessels (protein low-bind) in order to carry out the binding demonstration:
Addition of 1 ml of ethanol for degreasing
Centrifugation, removal of ethanol and washing of the hair with H₂O
Addition of 1 ml of 1% BSA in PBS and incubation for 1 h at room temperature, gentle swirling movements.
Centrifugation, removal of the supernatant
Addition of the keratin-binding domains to be tested (coupled to tag—e.g. His₆, HA etc.) or control protein in 1 ml of PBS/0.05% Tween 20; incubation for 16 h at 4° C. (or at least 2 h at room temperature) with gentle swimming movements.
Centrifugation, removal of the supernatant
3× washing with PBS/0.05% Tween 20
Incubation with 1 ml monoclonal mouse anti-tag (His6 or HA) antibodies with peroxidase conjugate (1:2000 in PBS/0.05% Tween 20) [Monoclonal AntipolyHistidine Peroxidase Conjugate, produced in mouse, lyophilized powder, Sigma] for 2-4 h at room temperature, gentle swinging movement
3× washing with PBS/0.05% Tween 20
Addition of peroxidase substrate (1 ml/Eppendorf vessel)
Allow reaction to proceed until blue coloration (about 2 minutes).
Stop the reaction with 100 μl of 2 M H₂SO₄.
The absorption is measured at 405 nm.

### Peroxidase Substrate (Prepare Shortly Beforehand):

0.1 ml TMB solution (42 mM TMB in DMSO)
+10 ml of substrate buffer (0.1 M sodium acetate pH 4.9)
+14.7 μl H₂O₂ 3% strength

### BSA=Bovine serum albumin

### PBS=Phosphate buffered salt solution

### Tween 20=polyoxyethylene sorbitan monolaurate, n about 20

### TMB=3,5,3′,5′-tetramethylbenzidine

A binding test on hair carried out by way of example for KBD-B demonstrated considerable superiority of the binding of KBD-B to hair compared with significantly poorer binding of the comparison protein YaaD:

TABLE 1
Quantitative KBD activity test Hair:

| | | |
|---|---|---|
| 1 | Buffer | A_{405 nm} = 0.000 |
| 2 | Comparison protein YaaD | A_{405 nm} = 0.088 |
| 3 | KBD-B denatured | A_{405 nm} = 0.254 |
| 4 | KBD-B renatured | A_{405 nm} = 1.591 |

1 buffer;
2 comparison protein YaaD;
3 KBD-B denatured;
4 KBD-B renatured. The table shows the measured absorption values at 405 nm.

### EXAMPLE 10A

### Coupling of a Dye to KBD-B

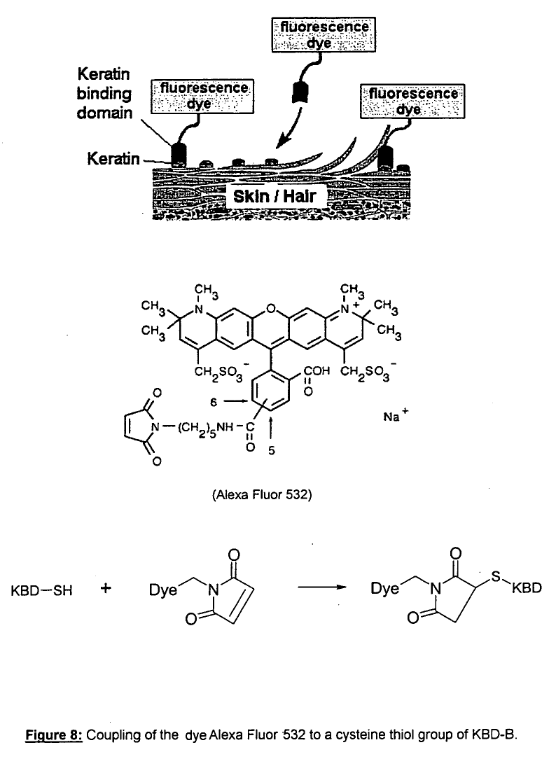

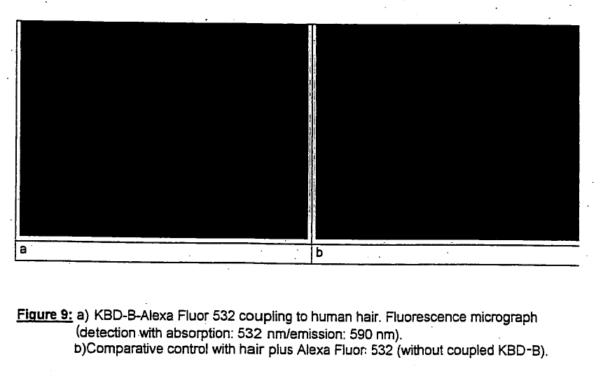

In order to couple a fluorescent dye (Alexa Fluor 532, Molecular Probes/Invitrogen) to the KBD-B protein, the dye was coupled via a maleic acid diimide linker to a cysteine thiol group by the following protocol. The reaction is depicted in FIG. 8.
1 mg of Alexa Fluor 532 was dissolved in 150 μl of PBS buffer of pH 7.0; this was followed by brief centrifugation to remove any undissolved constituents
10 μl of dissolved dye were added to 100 μg of KBD-B (1 mg/ml)
The mixture was incubated covered with Al foil, on a shaker at 450 pm and at 24° C. for 1 hour
10 μl of 1MDTT were added to inactivate the maleic acid diimide function of unreacted Alexa Fluor 532
Incubation was then carried out at 450 pm and at 24° C. (covered with Al foil) for 30 minutes

Coupling of KBD-B with coupled Alexa Fluor 532 to skin/hair can be determined by an activity test (see example 9 and 10). The KBD-B-Alexa Fluor 532 coupling which is bound to skin or hair in analogy to example 9 or 10 can be detected very easily on hair under the fluorescence microscope (detection with absorption: 532 nm/emission: 590 nm, see FIG. 9) or with the naked eye on bleached hair.

### EXAMPLE 11

### Use of the KBD in an Emulsion for Daycare

### O/W Type

### Al 1%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 67.8 | Aqua dem. |
| C | 4.0 | Caprylic/Capric Triglyceride, Sodium Acrylates |
| | | Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, |
| | | Tocopherol, Retinol |
| | 1.0 | Aqueous solution with about 5% keratin-binding domain |
| | | active ingredient |
| E | q.s. | Sodium Hydroxide |

### Al 5%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 63.8 | Aqua dem. |
| C | 4.0 | Caprylic/Capric Triglyceride, Sodium Acrylates |
| | | Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, |
| | | Tocopherol, Retinol |
| | 5.0 | Aqueous solution with about 5% keratin-binding domain |
| | | active ingredient |
| E | q.s. | Sodium Hydroxide |

Preparation: Heat phases A and B separately from one another to about 80° C. Stir Phase B into phase A and homogenize. Stir phase C into the combined phases A and B and homogenize again. Cool with stirring to about 40° C., add phase D, adjust the pH to about 6.5 using phase E, homogenize and cool to room temperature with stirring.
Note: The formulation is prepared without protective gas. Bottling must take place into oxygen-impermeable packagings, e.g. aluminum tubes.

### EXAMPLE 12

### Use of the KBD in a protective day cream

### O/W type

### Al 1%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 68.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | Aqueous solution with about 5% keratin-binding domain |
| | | active ingredient |
| E | q.s. | Sodium Hydroxide |

### Al 5%:

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 64.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | Aqueous solution with about 5% keratin-binding domain |
| | | active ingredient |
| E | q.s. | Sodium Hydroxide |

Preparation: Heat phases A and B separately from one another to about 80° C. Stir phase B into phase A and homogenize. Incorporate phase C into the combined phases A and B and homogenize. Cool with stirring to about 40° C. Add phase D, adjust the pH to about 6.5 using phase E and homogenize. Cool to room temperature with stirring.

### EXAMPLE 13

### Use of the KBD in a face-cleansing lotion

### O/W type

### Al 1%:

| | % | Ingredient (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Aqueous solution with about 5% keratin-binding domain |
| | | active ingredient |
| | 60.7 | Aqua dem. |

### Al 5%:

| | % | Ingredient (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Aqueous solution with about 5% keratin-binding domain |
| | | active ingredient |
| | 56.7 | Aqua dem. |

Preparation: Dissolve phase A. Stir phase B into phase A. Incorporate phase C into the combined phases A and B. Dissolve phase D, stir into the combined phases A, B and C and homogenize. After-stir for 15 min.

### EXAMPLE 14

### Use of the KBD in a Daily Care Body Spray

### Al 1%:

| | % | Ingredient (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Aqueous solution with about 5% keratin-binding domain |
| | | active ingredient |
| | 59.2 | Alcohol |

### Al 5%:

| | % | Ingredient (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Aqueous solution with about 5% keratin-binding domain |
| | | active ingredient |
| | 55.2 | Alcohol |

### Preparation: Weigh in the components of phase A and dissolve until clear.

### EXAMPLE 15

### Use of the KBD in a Skin Care Gel

### Al 1%:

| | % | Ingredient (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 1.0 | Aqueous solution with about 5% keratin-binding domain |
| | | active ingredient |
| | 75.4 | Aqua dem. |
| C | 0.8 | Triethanolamine |

### Al 5%:

| | % | Ingredient (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 5.0 | Aqueous solution with about 5% keratin-binding domain |
| | | active ingredient |
| | 71.4 | Aqua dem. |
| C | 0.8 | Triethanolamine |

Preparation: Dissolve phase A until clear. Allow phase B to swell and neutralize with phase C. Stir phase A into the homogenized phase B and homogenize.

### EXAMPLE 16

### Use of the KBD in an after Shave Lotion

### Al 1%:

| | % | Ingredient (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 1.0 | Aqueous solution with about 5% keratin-binding domain |
| | | active ingredient |
| | 0.1 | Triethanolamine |
| | 63.5 | Aqua dem. |

### Al 5%:

| | % | Ingredient (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 5.0 | Aqueous solution with about 5% keratin-binding domain |
| | | active ingredient |
| | 0.1 | Triethanolamine |
| | 59.5 | Aqua dem. |

### Preparation: Mix the components of phase A. Dissolve phase B, incorporate into phase A and homogenize.

### EXAMPLE 17

### Use of the KBD in an after Sun Lotion

### Al 1%:

| | % | Ingredient (INCI) |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 1.0 | Aqueous solution with about 5% keratin-binding domain |
| | | active ingredient |
| | 63.2 | Aqua dem. |
| C | 0.2 | Triethanolamine |

### Al 5%:

| | % | Ingredient (INCI) |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 5.0 | Aqueous solution with about 5% keratin-binding domain |
| | | active ingredient |
| | 59.2 | Aqua dem. |
| C | 0.2 | Triethanolamine |

Preparation: Mix the components of phase A. Stir phase B into phase A with homogenization. Neutralize with phase C and homogenize again.

### EXAMPLE 18

### Use of the KBD in a Sunscreen Lotion

### Al 1%:

| | % | Ingredient (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 59.7 | Aqua dem. |
| D | 1.0 | Aqueous solution with about 5% keratin-binding domain |
| | | active ingredient |
| | 1.0 | Phenoxyethanol, Methylparaben, Ethylparaben, |
| | | Butylparaben, Propyl- |
| | | paraben, Isobutylparaben |
| | 0.3 | Bisabolol |

### Al 5%:

| | % | Ingredient (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 55.7 | Aqua dem. |
| D | 5.0 | Aqueous solution with about 5% keratin-binding domain |
| | | active ingredient |
| | 1.0 | Phenoxyethanol, Methylparaben, Ethylparaben, |
| | | Butylparaben, Propyl- |
| | | paraben, Isobutylparaben |
| | 0.3 | Bisabolol |

Preparation: Heat the components of phases A and B separately from one another to about 80° C. Stir phase B into phase A and homogenize. Heat phase C to about 80° C. and stir into the combined phases A and B with homogenization. Cool to about 40° C. with stirring, add phase D and homogenize again.

### EXAMPLE 19

### Use of the KBD in a Sunscreen Lotion

### O/W Type

### Al 1%:

| | % | Ingredient (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl |
| | | Taurate Copolymer, |
| | | Squalane, Polysorbate 60 |
| | 0.2 | Disodium EDTA |
| | 5.0 | Propylene Glycol |
| | 0.5 | Panthenol |
| | 60.9 | Aqua dem. |
| D | 1.0 | Aqueous solution with about 5% keratin-binding domain |
| | | active ingredient |
| | 0.5 | Phenoxyethanol, Methylparaben, Butylparaben, |
| | | Ethylparaben, Propyl- |
| | | paraben, Isopropylparaben |
| | 1.0 | Tocopheryl Acetate |
| | 0.2 | Bisabolol |

### Al 5%:

| | % | Ingredient (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl |
| | | Taurate Copolymer, Squalane, Polysorbate 60 |
| | 0.2 | Disodium EDTA |
| | 5.0 | Propylene Glycol |
| | 0.5 | Panthenol |
| | 56.9 | Aqua dem. |
| D | 5.0 | Aqueous solution with about 5% keratin-binding domain |
| | | active ingredient |
| | 0.5 | Phenoxyethanol, Methylparaben, Butylparaben, Ethylparaben, |
| | | Propyl-paraben, Isopropylparaben |
| | 1.0 | Tocopheryl Acetate |
| | 0.2 | Bisabolol |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### EXAMPLE 20

### Use of the KBD in a Sunscreen Lotion

### O/W Type

### Al 1%:

| | % | Ingredient (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Aqua dem. |
| D | 1.0 | Aqueous solution with about 5% keratin-binding domain |
| | | active ingredient |
| | 1.0 | Tocopheryl Acetate |
| | 0.2 | Bisabolol |
| | q.s. | Perfume oil |
| | q.s. | Preservative |

### Al 5%:

| | % | Ingredient (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Aqua dem. |
| D | 5.0 | Aqueous solution with about 5% keratin-binding |
| | | domain active ingredient |
| | 1.0 | Tocopheryl Acetate |
| | 0.2 | Bisabolol |
| | q.s. | Perfume oil |
| | q.s. | Preservative |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### EXAMPLE 21

### Use of the KBD in a Foot Balsam

### Al 1%:

| | % | Ingredient (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | q.s. | Preservative |
| C | 1.0 | Bisabolol |
| | 1.0 | Tocopheryl Acetate |
| D | 1.0 | Aqueous solution with about 5% keratin-binding |
| | | domain active ingredient |
| | 5.0 | Witch Hazel Extract |

### Al 5%:

| | % | Ingredient (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | q.s. | Preservative |
| C | 1.0 | Bisabolol |
| | 1.0 | Tocopheryl Acetate |
| D | 5.0 | Aqueous solution with about 5% keratin-binding |
| | | domain active ingredient |
| | 5.0 | Witch Hazel Extract |

Preparation: Heat the components of phases A and B separately from one another to about 80° C. Stir phase B into phase A with homogenization. Cool to about 40° C. with stirring, add phases C and D and briefly after-homogenize. Cool to room temperature with stirring.

### EXAMPLE 22

### Use of the KBD in a W/O Emulsion with Bisabolol

### Al 1%:

| | % | Ingredient (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Aqua dem. |
| C | 1.0 | Aqueous solution with about 5% keratin-binding |
| | | domain active ingredient |
| | 0.5 | Tocopheryl Acetate |
| | 0.6 | Bisabolol |

### Al 5%:

| | % | Ingredient (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Aqueous solution with about 5% keratin-binding |
| | | domain active ingredient |
| | 0.5 | Tocopheryl Acetate |

Preparation: Heat phases A and B separately from one another to about 85° C. Stir phase B into phase A and homogenize. Cool to about 40° C. with stirring, add phase C and briefly homogenize again. Cool to room temperature with stirring. List of formulations for patent keratin-binding domain—haircare

### EXAMPLE 23

### Foam Conditioner with Setting Agent

### Al 1%

| | % | Ingredient (INCI) |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 1.0 | Aqueous solution with about 5% keratin-binding |
| | | domain active ingredient |
| | 68.1 | Aqua dem. |
| | 10.0 | Propane/Butane |

### Al 5%

| | % | Ingredient (INCI) |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 5.0 | Aqueous solution with about 5% keratin-binding |
| | | domain active ingredient |
| | 64.1 | Aqua dem. |
| | 10.0 | Propane/Butane |

### Preparation: Weigh the components of phase A together, stir until everything has dissolved and bottle.

### EXAMPLE 24

### Foam Conditioner

### Al 1%

| | % | Ingredient (INCI) |
|---|---|---|
| A | 1.0 | Polyquaternium-4 |
| | 0.5 | Hydroxyethyl Cetyldimonium Phosphate |
| | 1.0 | Aqueous solution with about 5% keratin-binding |
| | | domain active ingredient |
| | q.s. | Perfume oil |
| | q.s. | Preservative |
| | 91.5 | Aqua dem. |
| | 6.0 | Propane/Butane |

### Al 5%

| | % | Ingredient (INCI) |
|---|---|---|
| A | 1.0 | Polyquaternium-4 |
| | 0.5 | Hydroxyethyl Cetyldimonium Phosphate |
| | 5.0 | Aqueous solution with about 5% keratin-binding |
| | | domain active ingredient |
| | q.s. | Perfume oil |
| | q.s. | Preservative |
| | 87.5 | Aqua dem. |
| | 6.0 | Propane/Butane |

### Preparation: Weigh the components of phase A together, stir until everything has dissolved to give a clear solution and bottle.

### EXAMPLE 25

### Foam Conditioner

### Al 1%

| | % | Ingredient (INCI) |
|---|---|---|
| A | 1.0 | Polyquaternium-11 |
| | 0.5 | Hydroxyethyl Cetyldimonium Phosphate |
| | 1.0 | Aqueous solution with about 5% keratin-binding |
| | | domain active ingredient |
| | q.s. | Perfume oil |
| | q.s. | Preservative |
| | 91.5 | Aqua dem. |
| | 6.0 | Propane/Butane |

### Al 5%

| | % | Ingredient (INCI) |
|---|---|---|
| A | 1.0 | Polyquaternium-11 |
| | 0.5 | Hydroxyethyl Cetyldimonium Phosphate |
| | 5.0 | Aqueous solution with about 5% keratin-binding |
| | | domain active ingredient |
| | q.s. | Perfume oil |
| | q.s. | Preservative |
| | 87.5 | Aqua dem. |
| | 6.0 | Propane/Butane |

### Preparation: Weigh the components of phase A together, stir until everything has dissolved to give a clear solution and bottle.

### EXAMPLE 26

### Styling Foam

### Al 1%

| | % | Ingredient (INCI) |
|---|---|---|
| A | 0.5 | Laureth-4 |
| | q.s. | Perfume oil |
| B | 77.3 | Aqua dem. |
| | 10.0 | Polyquaternium-28 |
| | 1.0 | Aqueous solution with about 5% keratin-binding |
| | | domain active ingredient |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

### Al 5%

| | % | Ingredient (INCI) |
|---|---|---|
| A | 0.5 | Laureth-4 |
| | q.s. | Perfume oil |
| B | 73.3 | Aqua dem. |
| | 10.0 | Polyquaternium-28 |
| | 5.0 | Aqueous solution with about 5% keratin-binding |
| | | domain active ingredient |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### EXAMPLE 27

### Styling Foam

### Al 1%

| | % | Ingredient (INCI) |
|---|---|---|
| A | 2.0 | Cocotrimonium Methosulfate |
| | q.s. | Perfume oil |
| B | 78.5 | Aqua dem. |
| | 6.7 | Acrylates Copolymer |
| | 0.6 | AMP |
| | 1.0 | Aqueous solution with about 5% keratin-binding domain |
| | | active ingredient |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

| | % | Ingredient (INCI) |
|---|---|---|
| A | 2.0 | Cocotrimonium Methosulfate |
| | q.s. | Perfume oil |
| B | 74.5 | Aqua dem. |
| | 6.7 | Acrylates Copolymer |
| | 0.6 | AMP |
| | 5.0 | Aqueous solution with about 5% keratin-binding domain |
| | | active ingredient |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### EXAMPLE 28

### Styling Foam

### Al 1%

| | % | Ingredient (INCI) |
|---|---|---|
| A | 2.0 | Cocotrimonium Methosulfate |
| | q.s. | Perfume oil |
| B | 7.70 | Polyquaternium-44 |
| | 1.0 | Aqueous solution with about 5% keratin-binding domain |
| | | active ingredient |
| | q.s. | Preservative |
| | 79.3 | Aqua dem. |
| C | 10.0 | Propane/Butane |

### Al 5%

| | % | Ingredient (INCI) |
|---|---|---|
| A | 2.0 | Cocotrimonium Methosulfate |
| | q.s. | Perfume oil |
| B | 7.70 | Polyquaternium-44 |
| | 5.0 | Aqueous solution with about 5% keratin-binding domain |
| | | active ingredient |
| | q.s. | Preservative |
| | 75.3 | Aqua dem. |
| C | 10.0 | Propane/Butane |

### Preparation: Mix the components of phase A. Dissolve the components of phase B until clear, then stir phase B into phase A. Adjust the pH to 6-7, bottle with phase C.

### EXAMPLE 29

### Styling Foam

### Al 1%

| | % | Ingredient (INCI) |
|---|---|---|
| A | 2.00 | Cocotrimonium Methosulfate |
| | q.s. | Perfume oil |
| B | 72.32 | Aqua dem. |
| | 2.00 | VP/Acrylates/Lauryl Methacrylate Copolymer |
| | 0.53 | AMP |
| | 1.00 | Aqueous solution with about 5% keratin-binding domain |
| | | active ingredient |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

### Al 5%

| | % | Ingredient (INCI) |
|---|---|---|
| A | 2.00 | Cocotrimonium Methosulfate |
| | q.s. | Perfume oil |
| B | 68.32 | Aqua dem. |
| | 2.00 | VP/Acrylates/Lauryl Methacrylate Copolymer |
| | 0.53 | AMP |
| | 5.00 | Aqueous solution with about 5% keratin-binding domain |
| | | active ingredient |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture of A and B, then adjust the pH to 6-7. Bottle with phase D.

### EXAMPLE 30

### Styling Foam

### Al 1%

| | % | Ingredient (INCI) |
|---|---|---|
| A | 2.00 | Cetrimonium Chloride |
| | q.s. | Perfume oil |
| B | 67.85 | Aqua dem. |
| | 7.00 | Polyquaternium-46 |
| | 1.00 | Aqueous solution with about 5% keratin-binding domain |
| | | active ingredient |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

### Al 5%

| | % | Ingredient (INCI) |
|---|---|---|
| A | 2.00 | Cetrimonium Chloride |
| | q.s. | Perfume oil |
| B | 63.85 | Aqua dem. |
| | 7.00 | Polyquaternium-46 |
| | 5.00 | Aqueous solution with about 5% keratin-binding domain |
| | | active ingredient |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture of A and B, then adjust the pH to 6-7. Bottle with phase D.

### EXAMPLE 31

### Styling Foam

### Al 1%

| | % | Ingredient (INCI) |
|---|---|---|
| A | q.s. | PEG-40 Hydrogenated Castor Oil |
| | q.s. | Perfume oil |
| | 85.5 | Aqua dem. |
| B | 7.0 | Sodium Polystyrene Sulfonate |
| | 1.0 | Aqueous solution with about 5% keratin-binding domain |
| | | active ingredient |
| | 0.5 | Cetrimonium Bromide |
| | q.s. | Preservative |
| C | 6.0 | Propane/Butane |

### Styling Foam

### Al 5%

| | % | Ingredient (INCI) |
|---|---|---|
| A | q.s. | PEG-40 Hydrogenated Castor Oil |
| | q.s. | Perfume oil |
| | 81.5 | Aqua dem. |
| B | 7.0 | Sodium Polystyrene Sulfonate |
| | 5.0 | Aqueous solution with about 5% keratin-binding domain |
| | | active ingredient |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### EXAMPLE 32

### Styling Foam

### Al 1%

| | % | Ingredient (INCI) |
|---|---|---|
| A | q.s. | PEG-40 Hydrogenated Castor Oil |
| | q.s. | Perfume oil |
| | 92.0 | Aqua dem. |
| B | 0.5 | Polyquaternium-10 |
| | 1.0 | Aqueous solution with about 5% keratin-binding domain |
| | | active ingredient |
| | 0.5 | Cetrimonium Bromide |
| | q.s. | Preservative |
| C | 6.0 | Propane/Butane |

### Al 5%

| | % | Ingredient (INCI) |
|---|---|---|
| A | q.s. | PEG-40 Hydrogenated Castor Oil |
| | q.s. | Perfume oil |
| | 88.0 | Aqua dem. |
| B | 0.5 | Polyquaternium-10 |
| | 5.0 | Aqueous solution with about 5% keratin-binding domain |
| | | active ingredient |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### EXAMPLE 32

### Styling Foam

### Al 1%

| | % | Ingredient (INCI) |
|---|---|---|
| A | q.s. | PEG-40 Hydrogenated Castor Oil |
| | q.s. | Perfume oil |
| | 82.5 | Aqua dem. |
| B | 10.0 | Polyquaternium-16 |
| | 1.0 | Aqueous solution with about 5% keratin-binding domain |
| | | active ingredient |
| | 0.5 | Hydroxyethyl Cetyldimonium Phosphate |
| | q.s. | Preservative |
| C | 6.0 | Propane/Butane |

### Al 5%

| | % | Ingredient (INCI) |
|---|---|---|
| A | q.s. | PEG-40 Hydrogenated Castor Oil |
| | q.s. | Perfume oil |
| | 78.5 | Aqua dem. |
| B | 10.0 | Polyquaternium-16 |
| | 5.0 | Aqueous solution with about 5% keratin-binding domain |
| | | active ingredient |
| | 0.5 | Hydroxyethyl Cetyldimonium Phosphate |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### EXAMPLE 33

### Styling Foam

### Al 1%

| | % | Ingredient (INCI) |
|---|---|---|
| A | 2.0 | Cocotrimonium Methosulfate |
| | q.s. | Perfume oil |
| B | 84.0 | Aqua dem. |
| | 2.0 | Chitosan |
| | 1.0 | Aqueous solution with about 5% keratin-binding domain |
| | | active ingredient |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

### Al 5%

| | % | Ingredient (INCI) |
|---|---|---|
| A | 2.0 | Cocotrimonium Methosulfate |
| | q.s. | Perfume oil |
| B | 80.0 | Aqua dem. |
| | 2.0 | Chitosan |
| | 5.0 | Aqueous solution with about 5% keratin-binding |
| | | domain active ingredient |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Preparation: Mix the components of phase A. Add the components of phase B one after the other and dissolve. Bottle with phase C.

### EXAMPLE 34

### Care Shampoo

### Al 1%

| | % | Ingredient (INCI) |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 1.0 | Aqueous solution with about 5% keratin-binding |
| | | domain active ingredient |
| | 7.7 | Polyquaternium-44 |
| | 2.0 | Amodimethicone |
| | q.s. | Perfume oil |
| | q.s. | Preservative |
| | 1.0 | Sodium Chloride |
| | 43.3 | Aqua dem. |
| B | q.s. | Citric Acid |

### Al 5%

| | % | Ingredient (INCI) |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, |
| | | Cocamide MEA, Laureth-10 |
| | 5.0 | Aqueous solution with about 5% keratin-binding |
| | | domain active ingredient |
| | 7.7 | Polyquaternium-44 |
| | 2.0 | Amodimethicone |
| | q.s. | Perfume oil |
| | q.s. | Preservative |
| | 1.0 | Sodium Chloride |
| | 39.3 | Aqua dem. |
| B | q.s. | Citric Acid |

Preparation: Mix the components of phase A and dissolve. Adjust the pH to 6-7 with citric acid.

### EXAMPLE 35

### Shower Gel

### Al 1%

| | % | Ingredient (INCI) |
|---|---|---|
| A | 40.0 | Sodium Laureth Sulfate |
| | 5.0 | Decyl Glucoside |
| | 5.0 | Cocamidopropyl Betaine |
| | 1.0 | Aqueous solution with about 5% keratin-binding |
| | | domain active ingredient |
| | 1.0 | Panthenol |
| | q.s. | Perfume oil |
| | q.s. | Preservative |
| | 2.0 | Sodium Chloride |
| | 46.0 | Aqua dem. |
| B | q.s. | Citric Acid |

### Al 5%

| | % | Ingredient (INCI) |
|---|---|---|
| A | 40.0 | Sodium Laureth Sulfate |
| | 5.0 | Decyl Glucoside |
| | 5.0 | Cocamidopropyl Betaine |
| | 5.0 | Aqueous solution with about 5% keratin-binding |
| | | domain active ingredient |
| | 1.0 | Panthenol |
| | q.s. | Perfume oil |
| | q.s. | Preservative |
| | 2.0 | Sodium Chloride |
| | 42.0 | Aqua dem. |
| B | q.s. | Citric Acid |

Preparation: Mix the components of phase A and dissolve. Adjust the pH to 6-7 with citric acid.

### EXAMPLE 36

### Shampoo

### Al 1%

| | % | Ingredient (INCI) |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 44.6 | Aqua dem. |
| | 1.0 | Aqueous solution with about 5% keratin-binding |
| | | domain active ingredient |
| | 0.3 | Polyquaternium-10 |
| | 1.0 | Panthenol |
| | q.s. | Preservative |
| | 1.0 | Laureth-3 |
| | 2.0 | Sodium Chloride |

### Al 5%

| | % | Ingredient (INCI) |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 40.6 | Aqua dem. |
| | 5.0 | Aqueous solution with about 5% keratin-binding |
| | | domain active ingredient |
| | 0.3 | Polyquaternium-10 |
| | 1.0 | Panthenol |
| | q.s. | Preservative |
| | 1.0 | Laureth-3 |
| | 2.0 | Sodium Chloride |

Preparation: Mix the components of phase A and dissolve. Adjust the pH to 6-7 with citric acid.

### EXAMPLE 37

### Shampoo

### Al 1%

| | % | Ingredient (INCI) |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.00 | Aqueous solution with about 5% keratin-binding |
| | | domain active ingredient |
| | 0.15 | Guar Hydroxypropyltrimonium Chloride |
| | 2.00 | Laureth-3 |
| | 58.00 | Aqua dem. |
| | q.s. | Citric Acid |
| B | 3.00 | PEG-150 Distearate |

### Al 5%

| | % | Ingredient (INCI) |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 5.00 | Aqueous solution with about 5% keratin-binding |
| | | domain active ingredient |
| | 0.15 | Guar Hydroxypropyltrimonium Chloride |
| | 2.00 | Laureth-3 |
| | 54.00 | Aqua dem. |
| | q.s. | Citric Acid |
| B | 3.00 | PEG-150 Distearate |

Preparation: Weigh in the components of phase A and dissolve. Adjust the pH to 6-7. Add phase B and heat to about 50° C. Cool to room temperature with stirring.

### EXAMPLE 38

### Moisturizing Bodycare Cream

### Al 1%

| | % | Ingredient (INCI) |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | Aqueous solution with about 5% keratin-binding |
| | | domain active ingredient |
| | 1.0 | Panthenol |
| | 0.5 | Magnesium Aluminum Silicate |
| | q.s. | Preservative |
| | 65.5 | Aqua dem. |
| C | q.s. | Perfume oil |
| D | q.s. | Citric Acid |

### Al 5%

| | % | Ingredient (INCI) |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 5.0 | Aqueous solution with about 5% keratin-binding |
| | | domain active ingredient |
| | 1.0 | Panthenol |
| | 0.5 | Magnesium Aluminum Silicate |
| | q.s | Preservative |
| | 61.5 | Aqua dem. |
| C | q.s. | Perfume oil |
| D | q.s. | Citric Acid |

Preparation: Heat phases A and B separately to about 80° C. Briefly prehomogenize phase B, then stir phase B into phase A and homogenize again. Cool to about 40° C., add phase C and homogenize thoroughly again. Adjust the pH to 6-7 with citric acid.

### EXAMPLE 39

### Moisturizing Bodycare Cream

### Al 1%

| | % | Ingredient (INCI) |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 62.9 | Aqua dem. |
| C | q.s. | Perfume oil |
| | 1.0 | Aqueous solution with about 5% keratin-binding |
| | | domain active ingredient |

| | % | Ingredient (INCI) |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 58.9 | Aqua dem. |
| C | q.s. | Perfume oil |
| | 5.0 | Aqueous solution with about 5% keratin-binding |
| | | domain active ingredient |

Preparation: Heat phases A and B separately to about 80° C. Stir phase B into phase A and homogenize. Cool to about 40° C. with stirring, add phase C and homogenize again. Allow to cool to room temperature with stirring.

### EXAMPLE 40

### Liquid Make-Up

### O/W Type

### Al 1%

| | % | Ingredient (INCI) |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 61.9 | Aqua dem. |
| C | 0.1 | Bisabolol |
| | 1.0 | Aqueous solution with about 5% keratin-binding |
| | | domain active ingredient |
| | q.s. | Perfume oil |
| D | 5.7 | C.I. 77 891, Titanium Dioxide |
| | 1.1 | Iron Oxides |

### Al 5%

| | % | Ingredient (INCI) |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 57.9 | Aqua dem. |
| C | 0.1 | Bisabolol |
| | 5.0 | Aqueous solution with about 5% keratin-binding |
| | | domain active ingredient |
| | q.s. | Perfume oil |
| D | 5.7 | C.I. 77 891, Titanium Dioxide |
| | 1.1 | Iron Oxides |

Preparation: Heat phases A and B separately to about 80° C. Stir phase B into phase A and homogenize. Cool to about 40° C. with stirring, add phases C and D and thoroughly homogenize again. Allow to cool to room temperature with stirring.

### EXAMPLE 41

The active ingredient employed in the following exemplary formulations was a 5% by weight aqueous solution of a keratin-binding domain or of a keratin-binding effector molecule. The following data are parts by weight.

### Clear Shampoo

| Ingredients (INCI) | 1 | 2 | 3 | 4 | 5 |
|---|---|---|---|---|---|
| Texapon N 70 | 13.00 | 15.00 | 10.50 | 12.50 | 10.00 |
| Dehyaufn PK 45 | 7.50 | 7.00 | 5.00 | 5.50 | 10.00 |
| Cetiol HE | 2.00 | 2.50 | 3.50 | 5.00 | 2.30 |
| Fragance | 0.10 | 0.10 | 0.10 | 0.10 | 0.10 |
| Aqueous solution with | 1.0 | 5.0 | 0.1 | 0.5 | 10.0 |
| keratin-binding domain | | | | | |
| active ingredient | | | | | |
| D-Panthenol USP | 1.00 | 1.50 | 1.80 | 1.70 | 1.40 |
| Preservative | 0.10 | 0.10 | 0.10 | 0.10 | 0.10 |
| Citric Acid | 0.10 | 0.10 | 0.10 | 0.10 | 0.10 |
| Luviquat Ultra Care | 1.50 | 1.00 | 1.50 | 1.20 | 1.10 |
| Sodium Chloride | 1.50 | 1.40 | 1.40 | 1.30 | 1.50 |
| Aqua dem. | ad 100 | ad 100 | ad 100 | ad 100 | ad 100 |

### Shampoo

| Ingredients (INCI) | 1 | 2 | 3 | 4 | 5 |
|---|---|---|---|---|---|
| Texapon NSO | 35.00 | 40.00 | 30.00 | 45.00 | 27.00 |
| Plantacare 2000 | 5.00 | 5.50 | 4.90 | 3.50 | 7.00 |
| Tego Betain L7 | 10.00 | 5.00 | 12.50 | 7.50 | 15.00 |
| Fragrance | 0.10 | 0.10 | 0.10 | 0.10 | 0.10 |
| Aqueous solution with | 1.0 | 5.0 | 0.1 | 0.5 | 10.0 |
| keratin-binding domain | | | | | |
| active ingredient | | | | | |
| D-Panthenol USP | 0.50 | 1.00 | 0.80 | 1.50 | 0.50 |
| Preservative | 0.10 | 0.10 | 0.10 | 0.10 | 0.10 |
| Citric Acid | 0.10 | 0.10 | 0.10 | 0.10 | 0.10 |
| Rewopal LA 3 | 0.50 | 2.00 | 0.50 | 0.50 | 2.00 |
| Sodium Chloride | 1.50 | 1.50 | 1.50 | 1.50 | 1.50 |
| Aqua dem. | ad 100 | ad 100 | ad 100 | ad 100 | ad 100 |

### Clear Conditioner Shampoo

| Ingredients (INCI) | 1 | 2 | 3 | 4 | 5 |
|---|---|---|---|---|---|
| Amphotensid GB 2009 | 10.00 | 15.00 | 20.00 | 12.00 | 17.00 |
| Plantacare 2000 | 5.00 | 6.00 | 7.00 | 8.00 | 4.00 |
| Tego Betain L7 | 15.00 | 12.00 | 10.00 | 18.00 | 20.00 |
| Luviquat FC 550 | 0.30 | 0.20 | 0.20 | 0.20 | 0.30 |
| Fragrance | 0.10 | 0.10 | 0.10 | 0.10 | 0.10 |
| Aqueous solution with | 20.0 | 5.0 | 1.0 | 0.5 | 10.0 |
| keratin-binding domain | | | | | |
| active ingredient | | | | | |
| Cremophor PS 20 | 5.00 | 1.00 | 1.00 | 7.00 | 5.00 |
| Preservative | 0.10 | 0.10 | 0.10 | 0.10 | 0.10 |
| Rewopal LA 3 | 2.00 | 1.00 | 0.50 | 2.00 | 2.00 |
| Citric Acid | 0.20 | 0.20 | 0.20 | 0.20 | 0.20 |
| Stepan PEG-600 DS | 3.00 | 2.00 | 2.00 | 3.00 | 2.50 |
| Aqua dem. | ad 100 | ad 100 | ad 100 | ad 100 | ad 100 |

### Foam O/W Emulsions

| | Emulsion 1 | % by | Emulsion 2 | % by |
|---|---|---|---|---|
| | % by wt. | vol. | % by wt. | vol. |
| Stearic Acid | 5.00 | | 1.00 | |
| Cetyl Alcohol | 5.50 | | | |
| Cetearyl Alcohol | | | 2.00 | |
| PEG-40 Stearate | 8.50 | | | |
| PEG-20 Stearate | | | 1.00 | |
| Caprylic/Capric | 4.00 | | 2.00 | |
| Triglyceride | | | | |
| C12-15 Alkyl Benzoate | 10.00 | | 15.00 | |
| Cyclomethicone | 4.00 | | | |
| Dimethicone | | | 0.50 | |
| Aqueous solution with | 5.0 | | 10.0 | |
| keratin-binding domain | | | | |
| active ingredient | | | | |
| Octyl Isostearate | | | 5.00 | |
| Myristyl Myristate | | | 2.00 | |
| Ceresin | 1.50 | | | |
| Glycerin | | | 3.00 | |
| Filter | | | | |
| Hydroxypropyl Starch | 1.00 | | 3.50 | |
| Phosphate | | | | |
| BHT | | | 0.02 | |
| Disodium EDTA | 0.50 | | 0.10 | |
| Perfume, Preservative | q.s. | | q.s. | |
| Colorant | q.s. | | q.s. | |
| Potassium Hydroxide | q.s. | | q.s. | |
| Aqua dem. | ad 100 | | ad 100 | |
| | adjust pH | | adjust pH | |
| | to 6.5-7.5 | | to 5.0-6.0 | |
| Emulsion 1 | | 70 | | |
| Emulsion 2 | | | | 35 |
| Gas (nitrogen) | | 30 | | |
| Gas (helium) | | | | 65 |

### Conditioner Shampoo with Pearlescence

| | 1 | 2 | 3 |
|---|---|---|---|
| Polyquaternium-10 | 0.50 | 0.50 | 0.40 |
| Sodium Laureth Sulfate | 9.00 | 8.50 | 8.90 |
| Cocamidopropyl Betaine | 2.50 | 2.60 | 3.00 |
| Benzophenone-4 | 1.50 | 0.50 | 1.00 |
| Aqueous solution with keratin-binding | 1.0 | 5.0 | 0.5 |
| domain active ingredient | | | |
| Pearlescent aqueous solution with keratin- | 2.00 | 2.50 | |
| binding domain active ingredient | | | |
| Disodium EDTA | 0.10 | 0.15 | 0.05 |
| Preservative, Perfume, Thickener | q.s. | q.s. | q.s. |
| Aqua dem. | ad 100 | ad 100 | ad 100 |

adjust pH to 6.0

### Clear Conditioner Shampoo

| | 1 | 2 | 3 |
|---|---|---|---|
| Polyquaternium-10 | 0.50 | 0.50 | 0.50 |
| Sodium Laureth Sulfate | 9.00 | 8.50 | 9.50 |
| Aqueous solution with keratin-binding | 5.0 | 0.1 | 3.0 |
| domain active ingredient | | | |
| Benzophenone-3 | 1.00 | 1.50 | 0.50 |
| Imidosuccinic Acid, Na | 0.20 | 0.20 | 0.80 |
| Preservative, Perfume, Thickener | q.s. | q.s. | q.s. |
| Aqua dem. | ad 100 | ad 100 | ad 100 |

adjust pH to 6.0

Clear Conditioner Shampoo with Volume Effect

| | 1 | 2 | 3 |
|---|---|---|---|
| Sodium Laureth Sulfate | 10.00 | 10.50 | 11.00 |
| Ethylhexyl Methoxycinnamate | 2.00 | 1.50 | 2.30 |
| Aqueous solution with keratin-binding | 10.0 | 0.1 | 0.5 |
| domain active ingredient | | | |
| Cocamidopropyl Betaine | 2.50 | 2.60 | 2.20 |
| Disodium EDTA | 0.01 | 0.10 | 0.01 |
| Preservative, Perfume oil, Thickener | q.s. | q.s. | q.s. |
| Aqua dem. | ad 100 | ad 100 | ad 100 |

adjust pH to 6.0

### Gel Cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Polyacrylic Acid | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul A Plus ™ | 2.00 | 1.50 | 0.75 | 1.00 |
| UvaSorb K2A | | 3.00 | | |
| Ethylhexyl Methoxycinnamate | 3.00 | | 1.00 | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethylhexyl Triazone | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | 3.50 | 4.00 | 7.00 | 9.00 |
| Titanium Dioxide-microfine | 1.00 | | 3.00 | |
| Zinc Oxide-microfine | | | | 0.25 |
| Aqueous solution with keratin- | 0.1 | 0.5 | 1.0 | 0.02 |
| binding domain active ingredient | | | | |
| Cyclic Dimethylpolysiloxane | 5.00 | 5.50 | 5.00 | 5.50 |
| Dimethicone Polydimethylsiloxane | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume | 0.20 | | 0.20 | |
| Aqua dem. | ad 100 | ad 100 | ad 100 | ad 100 |

adjust pH to 6.0

### OW Sunscreen Formulation

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Aqueous solution with keratin- | 10.0 | 0.5 | 3.0 | 5.0 | 0.1 | 0.02 | 7.5 |
| binding domain active | | | | | | | |
| ingredient | | | | | | | |
| Uvinul A Plus ™ | 2.00 | 1.50 | 0.75 | 1.00 | 2.10 | 4.50 | 5.00 |
| UVASorb K2A | | | | | | | |
| Ethylhexyl Methoxycinnamate | | | | | 5.00 | 6.00 | 8.00 |
| Bis-Ethylhexyloxyphenol | | 1.50 | | 2.00 | 2.50 | | 2.50 |
| Methoxyphenyl Triazine | | | | | | | |
| Butyl | | | 2.00 | | 2.00 | 1.50 | |
| Methoxydibenzoylmethane | | | | | | | |
| Disodium Phenyl | 2.50 | | 0.50 | 2.00 | | 0.30 | |
| Dibenzimidazole Tetrasulfonate | | | | | | | |
| Ethyhexyl Triazone | 4.00 | | 3.00 | 4.00 | | 2.00 | |
| Octocrylene | | 4.00 | | | | | 7.50 |
| Diethylhexyl Butamido Triazone | 1.00 | | | 2.00 | 1.00 | | 1.00 |
| Phenylbenzimidazole Sulfonic | 0.50 | | 3.00 | | | | |
| Acid | | | | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide-microfine | 1.00 | | 3.00 | | 3.50 | | 1.50 |
| Zinc Oxide-microfine | | | | 0.25 | | 2.00 | |
| C12-15 Alkyl Benzoate | | 0.25 | | | 4.00 | 7.00 | |
| Dicaprylyl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethicone | 2.00 | | 0.50 | | 0.50 | | |
| Shea Butter | | 2.00 | | | | | |
| PVP Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Fucogel 1000 | | 3.00 | 10.00 | | | | |
| Glycine Soya (Soybean) Oil | | | | 0.50 | | 1.50 | 1.00 |
| Ethylhexyloxyglycine | 0.30 | | | | | | |
| DMDM Hydantaufin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic Acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Aqua dem. | ad 100 | ad 100 | ad 100 | ad 100 | ad 100 | ad 100 | ad 100 |

### Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Sodium Carbomer | | 0.20 | | 0.30 | |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Aqueous solution with keratin-binding | 5.0 | 0.5 | 3.0 | 0.1 | 10.0 |
| domain active ingredient | | | | | |
| Uvinul A Plus ™ | 2.00 | 1.50 | 0.75 | 1.00 | 2.10 |
| UVASorb K2A | | 3.50 | | | |
| Ethylhexyl Methoxycinnamate | | | | | 5.00 |
| Bis-Ethylhexyloxyphenol | | 1.50 | | 2.00 | 2.50 |
| Methoxyphenyl Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic | | 1.50 | | 1.00 | 1.00 |
| Acid | | | | | |
| Diethylhexyl 2,6-Naphthalate | | | 7.00 | | 9.00 |
| Titanium Dioxide-microfine | 1.00 | | 3.00 | | 3.50 |
| Zinc Oxide-microfine | | | | 0.25 | |
| C12-15 Alkyl Benzoate | 2.00 | 2.50 | | | |
| Dicaprylyl Ether | | 4.00 | | | |
| Butylene Glycol Dicaprylate/Dicaprate | 4.00 | | 2.00 | 6.00 | |
| Dicaprylyl Carbonate | | 2.00 | 6.00 | | |
| Dimethicone | | 0.50 | 1.00 | | |
| Phenyl Trimethicone | 2.00 | | 0.50 | | |
| Shea Butter | | 2.00 | | 5.00 | |
| PVP Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya I | | | 1.50 | | 1.00 |
| Vitamin E Acetate | 0.50 | | 0.25 | | 1.00 |
| Alpha Glucosilrutin | 0.60 | | | 0.25 | |
| Fucogel 1060 | | 2.50 | 0.50 | | 2.00 |
| DMDM Hydanaufin | | 0.60 | 0.45 | 0.25 | |
| Glyacil-S | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume | 0.20 | | 0.05 | 0.40 | |
| Aqua dem. | ad 100 | ad 100 | ad 100 | ad 100 | ad 100 |

WO Sunscreen Emulsion

| | 1 | 2 | 3 | 4 | 5 |
|---|---|---|---|---|---|
| Cetyldimethicone Copolyol | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Aqueous solution with keratin-binding | 5.0 | 1.0 | 10.0 | 0.5 | 0.1 |
| domain active ingredient | | | | | |
| Uvinul A Plus ™ | 2.00 | 1.50 | 0.75 | 1.00 | 2.10 |
| UVASorb K2A | | 2.00 | | | |
| Ethylhexyl Methoxycinnamate | | | | | 5.00 |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic | | 1.50 | | 1.00 | 1.00 |
| Acid | | | | | |
| Diethylhexyl 2,6-Naphthalate | | | 7.00 | | 4.00 |
| Titanium Dioxide-microfine | 1.00 | | 3.00 | | 3.50 |
| Zinc Oxide-microfine | | | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Shea Butter | | | 3.00 | | |
| Petrolatum | | 4.50 | | | |
| PVP Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soya | | 1.00 | 1.50 | | 1.00 |
| MgSO₄ | 1.00 | 0.50 | | 0.50 | |
| MgCl₂ | | | 1.00 | | 0.70 |
| Vitamin E Acetate | 0.50 | | 0.25 | | 1.00 |
| Ascorbyl Palmitate | 0.50 | | | 2.00 | |
| Fucogel 1000 | | | | 3.50 | 1.00 |
| DMDM Hydanaufin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume | 0.20 | | 0.40 | 0.35 | |
| Aqua dem. | ad 100 | ad 100 | ad 100 | ad 100 | ad 100 |

### Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Carnauba Wax | 1.50 | 2.00 | 2.00 | 1.50 |
| Cera Alba | 0.50 | 0.50 | 0.50 | 0.40 |
| C16-40 Alkyl Stearate | | 1.50 | 1.50 | 1.50 |
| Aqueous solution with keratin- | 0.5 | 3.0 | 1.0 | 5.0 |
| binding domain active | | | | |
| ingredient | | | | |
| Uvinul A Plus ™ | 2.00 | 1.50 | 0.75 | 9.00 |
| UVASorb K2A | | 2.00 | | 4.00 |
| Ethylhexyl Methoxycinnamate | | 3.00 | | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethyhexyl Triazone | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | | | 7.00 | |
| Titanium Dioxide-microfine | 1.00 | | 3.00 | |
| Zinc Oxide-microfine | | | | 0.25 |
| Vitamin E Acetate | 0.50 | 1.00 | | |
| Ascorbyl Palmitate | 0.05 | | 0.05 | |
| Buxux Chinensis | 2.00 | 1.00 | | 1.00 |
| Perfume, BHT | 0.10 | 0.25 | | 0.35 |
| Ricinus Communis Oil | ad 100 | ad 100 | ad 100 | ad 100 |

### PIT Emulsion

| | 1 | 2 | 3 | 4 | 5 | 6 | 7 | 8 |
|---|---|---|---|---|---|---|---|---|
| Glyceryl Monostearate | 0.50 | 2.00 | 3.00 | 5.00 | | 0.50 | 4.00 | |
| SE | | | | | | | | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2 | | | | 0.75 | 0.30 | | | |
| Aqueous solution with | 0.1 | 5.0 | 0.01 | 0.5 | 3.0 | 0.25 | 10.0 | 3.0 |
| keratin-binding domain | | | | | | | | |
| active ingredient | | | | | | | | |
| Uvinul A Plus ™ | 2.00 | 1.50 | 0.75 | 1.00 | 2.10 | 4.50 | 5.00 | 2.10 |
| UVASorb K2A | | | 4.00 | | | | 1.50 | |
| Ethylhexyl | | | | | 5.00 | 6.00 | 8.00 | 5.00 |
| Methoxycinnamate | | | | | | | | |
| Bis- | | 1.50 | | 2.00 | 2.50 | | 2.50 | 2.50 |
| Ethylhexyloxyphenol | | | | | | | | |
| Methoxyphenyl Triazine | | | | | | | | |
| Butyl | | | 2.00 | | 2.00 | 1.50 | | 2.00 |
| Methoxydibenzoyl- | | | | | | | | |
| methane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethyhexyl Triazone | 4.00 | | 3.00 | 4.00 | | 2.00 | | |
| Octocrylene | | 4.00 | | | | | 7.50 | |
| Diethylhexyl Butamido | 1.00 | | | 2.00 | 1.00 | | 1.00 | 1.00 |
| Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole | | | 0.50 | | | 1.00 | | |
| Trisiloxane | | | | | | | | |
| Terephthalidene | | 1.50 | | 1.00 | 1.00 | | 0.50 | 1.00 |
| Dicamphor Sulfonic | | | | | | | | |
| Acid | | | | | | | | |
| Diethylhexyl 2,6- | | | 7.00 | | 10.00 | 7.50 | | 8.00 |
| Naphthalate | | | | | | | | |
| Titanium Dioxide- | 1.00 | | 3.00 | | 3.50 | | 1.50 | 3.50 |
| microfine | | | | | | | | |
| Zinc Oxide-microfine | | | | 0.25 | | 2.00 | | |
| C12-15 Alkyl Benzoate | 3.50 | | | 6.35 | | | | 0.10 |
| Cocoglyceride | | 3.00 | | 3.00 | | | | 1.00 |
| Dicaprylyl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| Ethyl Galactomannan | | 0.50 | | | 2.00 | | | |
| Hydrogenated Coco- | | | | | 3.00 | 4.00 | | |
| Glycerides | | | | | | | | |
| Abil Wax 2440 | | | | | | 1.50 | 2.00 | |
| PVP Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Shea Butter | | 2.00 | | 3.60 | | 2.00 | | |
| Iodopropyl | 0.12 | | | | 0.20 | | | |
| Butylcarbamate | | | | | | | | |
| Fucogel 1000 | | | | 0.10 | | | | |
| DMDM Hydanaufin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ocaufxyglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

### Gel Cream

| | 1 | 2 | 3 | 4 |
|---|---|---|---|---|
| Acrylate/C10-30 Alkyl acrylate | 0.40 | 0.35 | 0.40 | 0.35 |
| Crosspolymer | | | | |
| Polyacrylic acid | 0.20 | 0.22 | 0.20 | 0.22 |
| Luvigel EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Titanium Dioxide-microfine | 1.00 | | 1.50 | |
| Zinc Oxide-microfine | | 2.00 | | 0.25 |
| Aqueous solution with keratin- | 0.5 | 10.0 | 3.0 | 5.0 |
| binding domain active | | | | |
| ingredient | | | | |
| Dihydroxyaceaufn | | | 3.00 | 5.00 |
| Cyclic Dimethylpolysiloxane | 5.00 | 5.50 | 5.00 | 5.50 |
| Dimethicone Polydimethylsiloxane | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume | 0.20 | | 0.20 | |
| Aqua dem. | ad 100 | ad 100 | ad 100 | ad 100 |
| adjust pH to 6.0 | | | | |

### OW Self-Tannings

### Formulations

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Aqueous solution with | 0.1 | 0.5 | 0.025 | 5.0 | 3.0 | 10.0 | 1.0 |
| keratin-binding domain | | | | | | | |
| active ingredient | | | | | | | |
| Dihydroxyaceaufn | | | 3.00 | 5.00 | | 4 | |
| Titanium Dioxide- | 1.00 | | | | 1.50 | | 1.50 |
| microfine | | | | | | | |
| Zinc Oxide-microfine | | | | 0.25 | | 2.00 | |
| C12-15 Alkyl Benzoate | | 0.25 | | | 4.00 | 7.00 | |
| Dicaprylyl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea Butter | | 2.00 | | | | | |
| PVP Hexadecene | 0.20 | | | 0.50 | | 1.00 | |
| Copolymer | | | | | | | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Fucogel 1000 | | 3.00 | 10.00 | | | | |
| Glycine Soya | | | | 0.50 | | 1.50 | 1.00 |
| Ethylhexyloxyglycine | 0.30 | | | | | | |
| DMDM Hydanaufin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic Acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Aqua dem. | ad 100 | ad 100 | ad 100 | ad 100 | ad 100 | ad 100 | ad 100 |

### OW Make Up

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Aqueous solution with | 3.0 | 5.0 | 2.0 | 0.5 | 1.0 | 5.0 | 10.0 |
| keratin-binding domain | | | | | | | |
| active ingredient | | | | | | | |
| Titanium Oxide | 10.00 | 12.00 | 9.00 | 8.50 | 11.00 | 9.50 | 10.00 |
| Iron Oxide | 2.00 | 4.00 | 3.00 | 5.00 | 3.40 | 6.00 | 4.40 |
| Zinc Oxide | | 4.00 | | 2.00 | | 3.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea Butter | | 2.00 | | | | | |
| PVP Hexadecene | 0.20 | | | 0.50 | | 1.00 | |
| Copolymer | | | | | | | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soya | | | | 0.50 | | 1.50 | 1.00 |
| Ethylhexyloxyglycine | 0.30 | | | | | | |
| DMDM Hydanaufin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic Acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Aqua dem. | ad 100 | ad 100 | ad 100 | ad 100 | ad 100 | ad 100 | ad 100 |

### Self-Tanning Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel EM | | 2.00 | | 2.50 | 2.00 |
| Acrylate/C10-30 Alkyl Acrylate | 0.50 | | 0.40 | 0.10 | 0.50 |
| Crosspolymer | | | | | |
| Xanthan Gum | | 0.30 | 0.15 | | |
| Aqueous solution with keratin-binding | 3.0 | 1.0 | 0.5 | 0.1 | 5.0 |
| domain active ingredient | | | | | |
| Dihydroxyaceaufn | | | 3.00 | 5.00 | |
| Uvinul A Plus ™ | 2.00 | 1.50 | 0.75 | 1.00 | 2.10 |
| Titanium Dioxide-microfine | 1.00 | | 1.00 | | 1.00 |
| Zinc Oxide-microfine | | 1.90 | | 0.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Shea Butter | | 2.00 | | 5.00 | |
| PVP Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya | | | 1.50 | | 1.00 |
| Vitamin E Acetate | 0.50 | | 0.25 | | 1.00 |
| Alpha-Glucosilrutin | 0.60 | | | 0.25 | |
| DMDM Hydanaufin | | 0.60 | 0.45 | 0.25 | |
| Glyacil-S | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume | 0.20 | | 0.05 | 0.40 | |
| Water | ad 100 | ad 100 | ad 100 | ad 100 | ad 100 |

### After-Sun Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylate/C10-30 Alkyl Acrylate | 0.50 | 0.30 | 0.40 | 0.10 | 0.50 |
| Crosspolymer | | | | | |
| Xanthan Gum | | 0.30 | 0.15 | | |
| Aqueous solution with keratin-binding | 0.1 | 5.0 | 0.5 | 3.0 | 1.0 |
| domain active ingredient | | | | | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya | | | 1.50 | | 1.00 |
| Vitamin E Acetate | 0.50 | | 0.25 | | 1.00 |
| Alpha-Glucosilrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume | 0.20 | | 0.05 | 0.40 | |
| Aqua dem. | ad 100 | ad 100 | ad 100 | ad 100 | ad 100 |

### WO Emulsions

| | 1 | 2 | 3 | 4 | 5 |
|---|---|---|---|---|---|
| Cetyl Dimethicone Copolyol | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Aqueous solution with keratin-binding | 5.0 | 10.0 | 0.1 | 0.5 | 1.0 |
| domain active ingredient | | | | | |
| Uvinul A Plus ™ | 2.00 | 1.50 | 0.75 | 1.00 | 2.10 |
| Titanium Dioxide-microfine | 1.00 | | 3.00 | | 3.50 |
| Zinc Oxide-microfine | | 0.90 | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Shea Butter | | | 3.00 | | |
| Petrolatum | | 4.50 | | | |
| PVP Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soya | | 1.00 | 1.50 | | 1.00 |
| MgSO₄ | 1.00 | 0.50 | | 0.50 | |
| MgCl₂ | | | 1.00 | | 0.70 |
| Vitamin E Acetate | 0.50 | | 0.25 | | 1.00 |
| Ascorbyl Palmitate | 0.50 | | | 2.00 | |
| Fucogel 1000 | | | | 3.50 | 7.00 |
| DMDM Hydanaufin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume | 0.20 | | 0.40 | 0.35 | |
| Aqua dem. | ad 100 | ad 100 | ad 100 | ad 100 | ad 100 |

### Solid Stabilized Emulsion

### (Pickering Emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric Triglyceride | 9.00 | 9.00 | 6.00 | | |
| C12-15 Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hecaufrite | 1.00 | 0.75 | | 0.50 | 0.25 |
| Cera Microcristallina + Paraffinum | | 0.35 | | | 5.00 |
| Liquidum | | | | | |
| Hydroxypropyl Methylcellulose | | | 0.10 | | 0.05 |
| Dimethicone | | | | | 3.00 |
| Aqueous solution with keratin-binding | 1.0 | 0.5 | 0.1 | 3.0 | 5.0 |
| domain active ingredient | | | | | |
| Titanium Dioxide + Alumina + Simethicone + Aqua | | 3.00 | | | |
| Titanium Dioxide + Trimethoxycaprylylsilane | | 2.00 | 4.00 | 2.00 | 4.00 |
| Silica Dimethyl Silylate | 2.50 | | | 6.00 | 2.50 |
| Boron Nitride | | | 1.00 | | |
| Starch/Sodium metaphosphate Polymer | 2.00 | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyethanol | | | 0.20 | | |
| Hexamidine Diisethionate | | | 0.40 | 0.50 | 0.40 |
| Diazolidinyl Urea | | | | | 0.08 |
| Ethanol | | | 0.23 | 0.20 | |
| Perfume | 5.00 | | 3.00 | 4.00 | |
| Aqua dem. | 0.20 | | 0.30 | 0.10 | |
| | ad 100 | ad 100 | ad 100 | ad 100 | ad 100 |

### Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Carnauba Wax | 1.50 | 2.00 | 2.00 | 1.50 |
| Cera Alba | 0.50 | 0.50 | 0.50 | 0.40 |
| C16-40 Alkyl Stearate | | 1.50 | 1.50 | 1.50 |
| Aqueous solution with keratin- | 10.0 | 1.0 | 3.0 | 0.1 |
| binding domain active | | | | |
| ingredient | | | | |
| Uvinul A Plus ™ | 2.00 | 1.50 | 0.75 | 9.00 |
| Titanium Dioxide-microfine | 1.00 | | 3.00 | |
| Zinc Oxide-microfine | | 1.00 | | 0.25 |
| Vitamin E Acetate | 0.50 | 1.00 | | |
| Ascorbyl Palmitate | 0.05 | | 0.05 | |
| Buxux Chinensis | 2.00 | 1.00 | | 1.00 |
| Perfume, BHT | 0.10 | 0.25 | | 0.35 |
| Ricinus Communis | ad 100 | ad 100 | ad 100 | ad 100 |

Self-Tanning PIT Emulsions

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyt Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 | | | | 0.75 | 0.30 | | | |
| Aqueous solution with | 0.1 | 0.5 | 0.01 | 5.0 | 0.5 | 3.0 | 0.025 | 10.0 |
| keratin-binding domain | | | | | | | | |
| active ingredient | | | | | | | | |
| Dihydroxyaceaufn | | | 3.00 | 5.00 | | | 4.00 | |
| Uvinul A Plus ™ | 2.00 | 1.50 | 0.75 | 1.00 | 2.10 | 4.50 | 5.00 | 2.10 |
| Titanium Dioxide-microfine | 1.00 | | 1.50 | | 3.50 | | 1.50 | 1.00 |
| Zinc Oxide-microfine | | 1.00 | | 0.25 | | 2.00 | | 1.50 |
| C12-15 Alkyl Benzoate | 3.50 | | | 6.35 | | | | 0.10 |
| Cocoglycerides | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| Ethyl Galacaufmannan | | 0.50 | | | 2.00 | | | |
| Hydrogenated Coco- | | | | | 3.00 | 4.00 | | |
| Glycerides | | | | | | | | |
| Abil Wax 2440 | | | | | | 1.50 | 2.00 | |
| PVP Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Shea Butter | | 2.00 | | 3.60 | | 2.00 | | |
| Iodopropyl Butylcarbamate | 0.12 | | | | 0.20 | | | |
| DMDM Hydanaufin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ocaufxyglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Myristyl Myristate | 3.50 | 3.00 | 4.00 | 3.00 |
| Benaufne-34 | 5.00 | 5.00 | 6.00 | 6.00 |
| Propylene Carbonate | 15.00 | 20.00 | 18.00 | 19.50 |
| Aqueous solution with keratin- | 1.0 | 0.5 | 3.0 | 5.0 |
| binding domain active ingredient | | | | |
| Vitamin E Acetate | 0.50 | 1.00 | | |
| Ascorbyl Palmitate | 0.05 | | 0.05 | |
| Buxux Chinensis | 2.00 | 1.00 | | 1.00 |
| Perfume, BHT | 0.10 | 0.25 | | 0.35 |
| Ricinus Communis | ad 100 | ad 100 | ad 100 | ad 100 |

Oil Gel

1. A cosmetic composition for treating keratin-containing materials, comprising at least one keratin-binding polypeptide sequence (i) in a cosmetically compatible medium.

2. The cosmetic composition according to claim 1, wherein the polypeptide sequence (i) has a binding affinity to human hair keratin, nail keratin, or skin keratin.

3. The cosmetic composition according to claim 1, wherein the polypeptide sequence (i) comprises at least one of the following polypeptide sequences,
(a) the polypeptide sequence of SEQ ID NO:1 at position 2193 to 2481
(b) the polypeptide sequence of SEQ ID NO:1 at position 2606 to 2871
(c) a polypeptide sequence which is modified compared with (a) in up to 60% of the amino acids,
(d) a polypeptide sequence which is modified compared with (b) in up to 50% of the amino acids,
with the proviso that the keratin binding of polypeptide sequence (c) or (d) amounts to at least 10% of the value shown by polypeptide sequence (a) or (b), measured in the test as in example 9 or 10.

4. The cosmetic composition according to claim 1, which comprises the polypeptide sequence (i) in an amount of from 0.01 to 30% by weight.

5. The cosmetic composition according to claim 1, which, besides the polypeptide sequence (i), comprises at least one cosmetic active ingredient.

6. The cosmetic composition according to claim 5, wherein the cosmetic active ingredient is chosen from the group consisting of natural or synthetic polymers, pigments, moisturizers, oils, waxes, enzymes, minerals, vitamins, sunscreens, dyes, fragrances, antioxidants and preservatives.

7. A composition for improving the combability of hair comprising the cosmetic composition according to claim 1.

8. A composition for improving the setting of hair comprising the cosmetic composition according to claim 1.

9. A composition for a conditioning effect on the skin comprising the cosmetic composition according to claim 1.

10. A composition for preparing compositions for skin, nail and hair cosmetics comprising the cosmetic composition according to claim 1.

11. A pharmaceutical composition for treating keratin-containing materials, comprising at least one keratin-binding polypeptide sequence (i) in a pharmaceutically compatible medium.

12. A keratin-binding effector molecule consisting of
(i) at least one polypeptide sequence which has a binding affinity for a keratin,
(ii) an effector molecule which is not naturally linked to the polypeptide sequence (i).

13. The keratin-binding effector molecule according to claim 12, where the polypeptide sequence (i) has binding affinity for the keratin of human hair, nails or skin.

14. The keratin-binding effector molecule according to claim 12, where the polypeptide sequence (i) includes at least one of the following polypeptide sequences,
(a) the polypeptide sequence of SEQ ID NO:1 at position 2269 to 2508
(b) the polypeptide sequence of SEQ ID NO:1 at position 2606 to 2871
(c) a polypeptide sequence which is modified compared with (a) in up to 70% of the amino acids,
(d) a polypeptide sequence which is modified compared with (b) in up to 70% of the amino acids,
with the proviso that the keratin binding of polypeptide sequence (c) or (d) amounts to at least 10% of the value shown by polypeptide sequence (a) or (b), measured in the assay described in example 9 or 10.

15. The keratin-binding effector molecule according to claim 12, where the effector molecule (ii) includes a polypeptide sequence.

16. The keratin-binding effector molecule according to claim 12, where the effector molecule (ii) has an enzymatic activity.

17. The keratin-binding effector molecule according to claim 12, where the effector molecule (ii) is a dye or a dye component.

18. The keratin-binding effector molecule according to claim 12, where the effector molecule (ii) is a UV filter.

19. The keratin-binding effector molecule according to claim 12, where the effector molecule (ii) is antioxidant.

20. The keratin-binding effector molecule according to claim 12, where the effector molecule (ii) is a carotenoid.

21. The keratin-binding effector molecule according to claim 12, where the effector molecule (ii) is a fungicide, insecticide or biocide.

22. The keratin-binding effector molecule according to claim 12, where the effector molecule (ii) is a vitamin or provitamin.

23. A composition for skin, nail and hair cosmetics comprising the keratin-binding effector molecules according to claim 12.

24. The keratin-binding effector molecule according to claim 12, where the effector molecule (ii) is attached to the polypeptide sequence (i) by covalent bonds.

25. The keratin-binding effector molecule according to claim 12, where the effector molecule (ii) is covalently bonded to functional groups of the side chain, the C-terminus or the N-terminus of the polypeptide (i), utilizing two chemical functions already present in (i) and (ii).

26. The keratin-binding effector molecule according to claim 12, where one or more effector molecules (ii) is/are covalently bonded to functional groups of the side chain, the C-terminus or the N-terminus of the polypeptide (i), by way of an at least bifunctional linker.

27. The keratin-binding effector molecule according to claim 12, where a spacer element is incorporated between effector molecule (ii) and polypeptide (i).

28. The keratin-binding effector molecule according to claim 26 which has a potential cleavage site for a protease, lipase, esterase, phosphatase or hydrolase between effector molecule (ii) and polypeptide (i).

29. The keratin-binding effector molecule according to claim 26 which has a further polypeptide sequence which permits easy purification of the fusion protein between effector molecule (ii) and polypeptide (i).

30. A process for preparing the keratin-binding polypeptide sequence (i) according to claim 3.

31. A process for preparing the keratin-binding effector molecule according to claim 14.


SEQUENCE LISTING (16 sequences)

SEQ ID NO: 1 - protein, 2871 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDRKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKVRNNYDEEIISLKNQFETEINITKTTIHQLTMQKEEDTSGYRAQIDNLTRENRSLSEEIKRLKNTLTQTTENLRRVEEDIQQQKATGSEVSQRKQQLEVELRQVTQMRTEESVRYKQSLDDAAKTIQDKNKEIERLKQLIDKETNDRKCLEDENARLQRVQYDLQKANSSATETINKLKVQEQELTRLRIDYERVSQERTVKDQDITRFQNSLKELQLQKQKVEEELNRLKRTASEDSCKRKKLEEELEGMRRSLKEQAIKITNLTQQLEQASIVKKRSEDDLRQQRDVLDGHLREKQRTQEELRRLSSEVEALRRQLLQEQESVKQAHLRNEHFQKAIEDKSRSLNESKIEIERLQSLTENLTKEHLMLEEELRNLRLEYDDLRRGRSEADSDKNATILELRSQLQISNNRTLELQGLINDLQRERENLRQEIEKFQKQALEASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATSVKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 2 - DNA, 23 bp, Unknown
  Oligonucleotide
ggtcagttacgtgcagctgaagg

SEQ ID NO: 3 - DNA, 18 bp, Unknown
  Oligonucleotide
gctgaggctgccggatcg

SEQ ID NO: 4 - DNA, 27 bp, Unknown
  Oligonucleotide
cgcgcctcgagccacatactggtctgc

SEQ ID NO: 5 - DNA, 23 bp, Unknown
  Oligonucleotide
gcttagctgaggctgccggatcg

SEQ ID NO: 6 - protein, 16 aa, Unknown
  N-terminal amino acids in expressed KBD-B
MRGSHHHHHHGSACEL

SEQ ID NO: 7 - protein, 10 aa, Unknown
  C-terminal amino acids in expressed KBD-B
GVDLQPSLIS

SEQ ID NO: 8 - DNA, 56 bp, Unknown
  Olionucleotide
gctaaggaattcaccatgcatcaccatcaccatcacgagccacatactggtctgct

SEQ ID NO: 9 - DNA, 35 bp, Unknown
  Oligonucleotide
gctggagaattctcagctaattaagcttggctgca

SEQ ID NO: 10 - DNA, 50 bp, Unknown
  Oligonucleotide
gctaaggaattccatcaccatcaccatcacgagccacatactggtctgct

SEQ ID NO: 11 - protein, 7 aa, Unknown
  N-terminal amino acids in expressed KBD-B
MHHHHHH

SEQ ID NO: 12 - protein, 99 aa, Unknown
  N-terminal amino acids in expressed KBD-B
MRFPSIFTAVLFAASSALAAPVNTTTEDETAQIPAEAVIGYSDLEGDFDVAVLPFSNSTNNGLLFINTTIASIAAKEEGVSLEKREAEAYVEFHHHHHH

SEQ ID NO: 13 - protein, 10 aa, Unknown
  N-terminal amino acids in expressed KBD-B
YVEFHHHHHH

SEQ ID NO: 14 - DNA, 45 bp, Unknown
  Oligonucleotide
caccatgcatcaccatcaccatcacgagccacatactggtctgct

SEQ ID NO: 15 - DNA, 20 bp, Unknown
  Oligonucleotide
gctaattaagcttggctgca

SEQ ID NO: 16 - protein, 75 aa, Unknown
  C-terminal amino acids in expressed KBD-B
KGGRADPAFLYKVVMIRLLTKPERKLLEGGPGTQLLFPLVRVNCALGVIMVIAVSCVKLLSAHNSTQHTSRKHKV